(19) United States
(12) Patent Application Publication
(10) Pub. No.: US 2026/0083126 A1
(43) Pub. Date: Mar. 26, 2026

(54) ANTIMICROBIAL COMPOSITIONS

(71) Applicant: AHV International B.V., Zwolle (NL)

(72) Inventors: Lex DE BOER, Wateringen (NL); Petrus Hendricus NIBBERING, Voorhout (NL); Anna Kristina WRONSKA, Braunschweig (DE); Shaista Sheroze KHOKHAR, Den Haag (NL)

(21) Appl. No.: 19/109,833

(22) PCT Filed: Sep. 8, 2023
(86) PCT No.: PCT/NL2023/050462

§ 371 Date: Mar. 7, 2025

(30) Foreign Application Priority Data
Sep. 8, 2022 (WO) PCT/NL2022/050507
Mar. 9, 2023 (NL) 2034306

(51) Int. Cl. A01N 41/12; A01P 1/00; A61K 31/10; A61L 2/00; A61L 101/32; A61P 31/04

(52) U.S. Cl. CPC A01N 41/12; A01P 1/00; A61K 31/10; A61L 2/18; A61L 2103/05; A61P 31/04; A61L 2101/32; A61L 2103/15

(57) ABSTRACT

The disclosure relates to organosulfur compounds and compositions comprising same. In particular, the disclosure relates to Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate. Such compositions are useful for treating microbial infection, in particular bacterial, fungal, rotozoan or algal infection. Compositions comprising said compounds are also provided for cleaning, disinfecting or agricultural use.

### FIELD OF THE INVENTION

The disclosure relates to organosulfur compounds and compositions comprising same. In particular, the disclosure relates to Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate. Such compositions are useful for treating microbial infection, in particular bacterial, fungal, protozoan or algal infection. Compositions comprising said compounds are also provided for cleaning, disinfecting or agricultural use.

### BACKGROUND OF THE INVENTION

Microbes, such as bacteria and fungi, are found almost everywhere and exist in very diverse forms. Most are not harmful and are actually indispensable for life on earth and essential for plant, animal and human health. For example, the microbiome in the intestines of humans and animals where bacteria and fungi live as symbionts with their host is the so-called gut flora. Also, bacteria are naturally present on the skin, which form part of the immune system. Another example is the soil biology, which for the most part consists of bacteria and fungi as well as of protozoa. Another example is the water biology and wastewater treatment where protozoa play an important role in enhancing clarity of the water. Some bacteria, fungi (including yeast), protozoa and algae can cause pathogenic infections, for example in animals, or humans. These pathological infections can lead to disease and illness of the infected individual. Plants are also susceptible to microbial infections.

Accordingly, a need exists for antimicrobial compounds as well as alternative treatments of microbial infections.

### SUMMARY OF THE INVENTION

The disclosure provides the following preferred embodiments.
1. A compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate, or a composition comprising at least one said compound for use in the treatment or prevention of a microbial infection, preferably a bacterial or a fungal infection.
2. A method of treating or preventing a microbial infection in an individual comprising administering to an individual in need thereof a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate, or a composition comprising at least one said compound.
3. A composition comprising a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate, wherein the composition is a pharmaceutical composition, a pesticide or a foodstuff composition.
4. An article having a surface at least partially coated with a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate or a composition comprising at least one said compound, preferably, wherein the article is a medical device or surgical device.
5. A cleaning or disinfecting composition comprising
(i) a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate; and
(ii) a surfactant.
6. An in vitro method comprising applying a cleaning or disinfecting composition to a surface, wherein said composition comprises a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate.
7. The method according to embodiment 6, wherein said method is a method of sanitizing or disinfecting said surface.
8. Use of a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate or a composition comprising at least one said compound, as a disinfectant, a sanitizing agent, or an antimicrobial agent in foodstuff.
9. A cleaning or disinfecting product comprising a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate or a composition comprising at least one said compound.
10. An agricultural composition comprising a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate and an agricultural acceptable excipient, carrier and/or solvent.
11. A method of preventing or treating an infection on a plant or plant part, comprising contacting a plant or plant part with a compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate or a composition comprising at least one said compound such that an infection is prevented or treated.
12. Use of the compound selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate or a composition comprising at least one said compound, for preventing or treating an infection on a plant or a plant part.
13. The use or method according to embodiments 11 or 12, wherein said compound or composition is applied directly to said plant, to the seed of said plant or to the soil of said plant or seed.
14. The use or method according to any of embodiments 11-13, wherein the plant is selected from the group consisting of Begoniaceae, Solanaceae, Amaranthaceae, Rosaceae and Brassicaceae, preferably wherein the plant is selected from the group consisting of Begonia, Tomato, Potato, Sugar beet, Strawberries, Cabbage, Apples, Orchidaceae, Chrysanthemum, Fabaceae, Cucurbitaceae, Pisum, Vitis, Vaccinia and Lactuca.

### BRIEF DESCRIPTION OF THE DRAWINGS

FIG. 1: Average MIC90 values per strain per compound. The values were determined during a MIC assay and then averaged per strain. Each data point is an average MIC90 per strain. The labels at the data points stand for the strain names E. coli=Ec, K. pneumoniae=Kp, S. agalactiae=Sag, L. garvieae=Lg, S. uberis=Su, S. suis=Ss, S. aureus=Sau, A. baumanni=Ab, M. lutues=MI, S. epidermidis=Se, P. aeruginosa=Pa, M. viscosa=MV, C. albicans=Ca. For bis-4-chlorophenyldisulfide and Lg, Kp, Su, Sag, Ec, Ss, Sau and Ab the MIC90 is represented as 1000 μM, but is actually >1000 μM, because no MIC90 value could be detected in the tested range. The same applies to Pa, Ab, Kp, Ec and Lg for bis-(4-fluorophenyl)-disulfide but then with the true MIC90 value laying above 4000 μM and for bis-(4-chlorophenyl) thiosulfonate and Ec, Kp, and Ab with MIC90 values >1000 μM.

FIG. 2: Cytotoxic effects of the used concentrations of emulsifiers Tween-80 and DMSO without antimicrobial (AM) compounds on Caco-2 cell line are presented.

FIG. 3: Cytotoxic effects of the used concentrations of emulsifiers Tween-80 and DMSO without antimicrobial (AM) compounds on HepG2 cell line are presented.

FIG. 4: Cytotoxic effect of emulsified AM2 on Caco-2 cells.

FIG. 5: Cytotoxic effect of emulsified AM2 on HepG2 cells.

FIG. 6: Cytotoxic effect of emulsified AM3 on Caco-2 cells.

FIG. 7: Cytotoxic effect of emulsified AM3 on HepG2 cells.

FIG. 8: Cytotoxic effect of emulsified AM6 on Caco-2 cells.

FIG. 9: Cytotoxic effect of emulsified AM6 on HepG2 cells.

FIG. 10: Cytotoxic effect of emulsified AM8-A on Caco-2 cells. AM8-A was emulsified with 30-times less Tween80 and no DMSO was added.

FIG. 11: Cytotoxic effect of emulsified AM8-A on HepG2 cells. AM8-A was emulsified with 30-times less Tween80 and no DMSO) was added.

FIG. 12: Cytotoxic effect of emulsified AM8-C on Caco-2 cells. AM8-C was emulsified with 30-times less Tween80 and no DMSO was added.

FIG. 13: Cytotoxic effect of emulsified AM8-C on HepG2 cells. AM8-(was emulsified with 30-times less Tween80 and no DMSO was added.

FIG. 14: Colony diameter of the tested fungi Pythium aphanidermatum after 7 days of incubation with AM2, bis(p-chlorophenyl) disulfide; AM3, bis(p-fluorophenyl) disulfide; AM6, bis(p-chlorophenyl) thiosulfonate; AM8, bis(iso-propyl) thiosulfonate and QQ22, diisopropyl thiosulfonate. The larger the colony diameter, the less growth inhibition was shown.

FIG. 15: Colony diameter of the tested fungi Phytophtora cinnamiomi after 7 days of incubation with AM2, AM3, AM6, AM8 and QQ2.

FIG. 16: Colony diameter of the tested fungi Fusarium oxysporum after 7 days of incubation with AM2, AM3, AM6, AM8 and QQ2.

FIG. 17: Colony diameter of the tested fungi Sclerotinia sclerotiorum after 7 days of incubation with AM2, AM3, AM6, AM8 and QQ2.

FIG. 18: Colony diameter of the tested fungi Rhizoctonia solani after 7 days of incubation with AM2, AM3, AM6, AM8 and QQ2.

FIG. 19: Colony diameter of the tested fungi Botrytis cinerea after 7 days of incubation with AM2, AM3, AM6, AM8 and QQ2.

FIG. 20: Raw data of colony diameter 3 days after inoculation.

FIG. 21: Raw data of colony diameter 7 days after inoculation.

FIG. 22: Raw data of colony diameter 10 days after inoculation.

FIG. 23: Raw data of colony diameter 14 days after inoculation.

FIG. 24: Effect of daily exposures to AM-8 on AMR Staphylococcus aureus within mature biofilms: Seven-day mature Staphylococcus aureus LUH14616 biofilms in 96-wells polystyrene plates were daily exposed for up to 4 days to various concentrations of AM-8 or—as control its diluent (1% DMSO)—prepared in PBS containing 2% v/v BHI. After each day the number of viable bacteria within the biofilm was enumerated microbiologically.

FIG. 25: Effect of AM-2 and AM-8 on AMR Staphylococcus aureus persisters: Seven day mature S. aureus LUH14616 biofilms were exposed for three days to 10× minimal bactericidal concentration (MBC) of rifampicin and ciprofloxacin daily. Next, the biofilms were exposed to various concentrations of AM-2 or AM-8 for 24 hrs or to 10×MBC rifampicin/ciprofloxacin (to demonstrate antibiotic tolerance of the persisters). The experiment was performed in triplicate.

FIG. 26: AM-8 induces little/no resistance in AMR S aureus LUH14616: Staphylococcus aureus LUH146161 were exposed to a subsequent range of AM-8 concentrations. For this purpose, two independently synthesized AM-8 batches were used with a minimal purity of 96.3%. In total 19 passages were run. As controls, S. aureus LUH 14616 were exposed to a dose-range of rifampicin and when resistance appeared to an adjusted range of concentrations of the antibiotics as indicated above for AM-8. Results are expressed as fold increase, i.e., the ratio between the MIC after the various passages relative to the MIC at the start of the experiment. The circles and squares coincide in the graph.
AM2=bis(4-chlorophenyl) disulfide
AM3=bis(4-fluorophenyl) disulfide
AM6=bis(4-chlorophenyl) thiosulfonate
AM8=diisopropyl thiosulfonate. AM8-A and AM8-C correspond to two separate batches of synthesized AM8.

### DETAILED DESCRIPTION OF THE DISCLOSED EMBODIMENTS

The disclosure provides novel uses and methods, as well as compositions comprising one or more compounds selected from Bis(4-fluorophenyl) disulfide, Bis(4-chlorophenyl) disulfide, Bis(4-chlorophenyl) thiosulfinate, Bis(4-fluorophenyl) thiosulfinate, Bis(4-chlorophenyl) thiosulfonate, Bis(4-fluorophenyl) thiosulfonate, and Di-isopropyl thiosulfonate. Said compounds are herein also described as “compounds of the invention”. Preferably, the compounds are selected from Bis(4-fluorophenyl) disulfide, Bis(4-chlorophenyl) disulfide, Bis(4-chlorophenyl) thiosulfonate, Bis(4-fluorophenyl) thiosulfonate, and Di-isopropyl thiosulfonate. Preferably the compounds are selected from Bis(4-fluorophenyl) disulfide, Bis(4-chlorophenyl) disulfide, Bis(4-chlorophenyl) thiosulfonate, and Di-isopropyl thiosulfonate.

In preferred embodiments, the compound is Bis(4-fluorophenyl) disulphide. In preferred embodiments, the compound is Bis(4-chlorophenyl) disulphide. In preferred embodiments, the compound is Bis(4-chlorophenyl) thiosulfinate. In preferred embodiments, the compound is Bis(4-fluorophenyl) thiosulfinate. In preferred embodiments, the compound is Bis(4-chlorophenyl) thiosulfonate. In preferred embodiments, the compound is Bis(4-fluorophenyl) thiosulfonate. In preferred embodiments, the compound is Di-isopropyl thiosulfonate.

It will be understood that the terms “sulphide” and “sulfide” are used interchangeably herein.

Methods for preparing the compounds referred to herein are known in the art. In some embodiments, the compounds are commercially available or can be prepared as described in example 1.

In some embodiments, compositions are provided wherein at least 50%, preferably at least 90% by weight of the active ingredients are compounds of the invention. In some embodiments, compositions are provided wherein the only active ingredients are compounds of the invention, optionally including further antimicrobial agents and/or anti-inflammatory agents. In some embodiments, compositions are provided wherein at least 50%, preferably at least 90% by weight of the active ingredients are compounds of the invention as disclosed herein. In some embodiments, compositions are provided wherein the only active ingredients are compounds of the invention, optionally including further antimicrobial agents and/or anti-inflammatory agents.

### Microbial Infection

The compounds disclosed herein and compositions comprising same are useful in the treatment or prevention of infection. For example, particular uses are for the treatment or prevention of respiratory infection, bowel infection, breast infection, udder infection, skin infection, bladder infection, ear infection, systemic infection, joint infection, brain infection. Suitable infections for treatment also include, for example, bacterial prostatitis, bacterial vaginosis, biliary tract infections, chronic sinusitis, chronic lung disease, dental caries, endocarditis, kidney stones, laryngitis, lung infection in cystic fibrosis, gingivitis mastitis, middle ear infections, nonsocomial (bloodstream) infections, obstructive pulmonary diseases, osteomyelitis, otitis media, periodontitis, pneumonia prostatitis, rhinosinusitis, sinusitis, tonsillitis, tuberculosis, urinary tract infections, and wound infections.

As used herein, “infection” refers to, e.g., pathogenic infections which can lead to disease. In particular, such infections are bacterial, fungal (including yeast), protozoan or algal infections. Preferably, the infection is a microbial infection. In a preferred embodiment, the infection is a bacterial infection. In a preferred embodiment, the infection is a fungal infection (including yeast infection). In a preferred embodiment, the infection is a protozoan infection. In a preferred embodiment, the infection is an algal infection.

### Treating or Preventing

As used herein, “treatment of infection” refers to a reduction in the severity and/or duration of the infection and/or a reduction of the severity and/or duration of symptoms from the infection. Preferably, said treatment results in restoration of the health of an individual. Preferably, the individual has less disease symptoms or for a shorter time. As used herein, “prevention of infection” refers to the prevention of or alternatively delaying the onset of infection or of one or more symptoms associated with infection.

The compounds disclosed herein are useful for treating an acute infection. Acute infections may be characterized by microorganisms, such as bacteria, growing in a planktonic state, whereas chronic infections are normally associated with the presence of biofilms. In some embodiments, an acute infection is characterized by infection (or symptoms of infection) of less than 6 months.

The compounds disclosed herein are also useful for treating a chronic and/or persistent infection. The terms persistent infection and chronic infection are often used interchangeably, but are based on different mechanisms. Persistent infections are normally held in check by immune defenses but may be activated when such immune defenses are weakened. Persistent infections are often asymptomatic and become clinically visible only when the immune defense fails to control the pathogen. Although persistent infections are often asymptomatic, a skilled person is well aware of means to detect such persistent infections, including e.g., detecting microorganisms from patient samples (e.g., blood or urine). In a chronic infection, pathogens remain in a group of cells/parts of tissue (e.g., joints or lung tissue). The patient always has symptoms of disease, although these might be milder that in the acute phase of infection.

In some embodiments, the infection is a chronic wound infection. In some embodiments, the wounds treated by the compounds of the invention comprise, for example, Staphylococcus aureus; Streptococci; gram negative bacteria, for example Treponema spp., Escherichia coli, Yersinia pestis, Pseudomonas aeruginosa; or yeast/fungi, for example Candida spp (albicans), Cladosporidium herbarum, Trichosporum, Rhodosporidium, and Malassezia.

In some embodiments, the infection is caused by a microorganism selected from one or more of Acinetobacter baumanni, Bacillus subtilis, Escherichia coli, Klebsiella pneumoniae, Piscirikettsia salmonis, Renibacterium salmoninarum, Staphylococcus aureus, Staphylococcus aureus (MRSA), Staphylococcus epidermidis, Streptococcus agalactiae, Escherichia coli, Clostridium perfringens, Moritella viscosa, Micrococcus luteus, Lactococcus garvieae, Candida albicans, Haemophilus influenzae, Streptococcus Suis, Streptococcus suis Type 2, Streptococcus uberis, Cutibacterium Agnes, Treponema spp, Yersinia pestis, Streptococcus dysgalactiae, Serratia marescens, Trueperella pyogenes, Mannheimia haemolytica, Pasteurella multocida, Pseudomonas aeruginosa, Burkolderia cepacia, Streptococcus neumoniae, Legionella neumophila, Fusobacterium necrophorum, Corynebacterium pseudotuberculosis, Streptococcus spp., Porphyromonas gingivalis, Pseudomonas aeruginosa, Enterococcus faecalis, Neisseria gonorrhoeae, Salmonella enteritidis and Pseudomonas aeruginosa. Preferably, the infection is caused by Candida albicans.

In preferred embodiments, the bacterial infection is caused by bacteria selected from one or more of Acinetobacter baumanni, Bacillus subtilis, Escherichia coli, Klebsiella pneumoniae, Piscirikettsia salmonis, Renibacterium salmoninarum, Staphylococcus aureus, Streptococcus agalactiae, Escherichia coli, Clostridium perfringens, Moritella viscosa, Micrococcus luteus, Lactococcus garvie, Candida albicans, Haemophilus influenzae, Streptococcus Suis Type 2, Streptococcus uberis and Cutibacterium Agnes.

In certain embodiments, the bacterial infection is caused by a Gram-negative bacterium. In certain embodiments, the bacterial infection is caused by a Gram-positive bacterium. In certain embodiments, the bacterial infection is caused by a multidrug-resistant bacterium. In certain embodiments, the bacterial infection is a methicillin-resistant Staphylococcus aureus (MRSA)-related infection or a Staphylococcus epidermidis (e.g., MRSE) related infection.

In a preferred embodiment, the infection causing bacteria is Escherichia coli, preferably the bacterial infection is recurrent urinary tract infection, catheter-associated urinary tract infection, or biliary tract infection.

In a preferred embodiment, the infection causing bacteria is Pseudomonas aeruginosa, preferably the bacterial infection is Cystic fibrosis lung infection, chronic wound infection, catheter-associated urinary tract infection, chronic rhinosinusitis, chronic otitis media, bronchiectasis, chronic obstructive pulmonary disease or contact lens-related keratitis.

In a preferred embodiment, the infection causing bacteria is Staphylococcus aureus, preferably the bacterial infection is Chronic osteomyelitis, chronic rhinosinusitis, endocarditis, chronic otitis media, or of (orthopaedic) implants.

In a preferred embodiment, the infection causing bacteria is Staphylococcus epidermidis, preferably the bacterial infection is Central venous catheter, orthopaedic implants, or chronic osteomyelitis.

In a preferred embodiment, the infection causing bacteria is Streptococcus pneumoniae, preferably the bacterial infection is infection of nasopharynx, chronic rhinosinositis, chronic otitis media, or infection in chronic obstructive pulmonary disease.

In a preferred embodiment, the infection causing bacteria is Streptococcus pyogenes, preferably the bacterial infection is infection of oral cavity and nasopharynx, recurrent tonsilitis.

In some embodiments, the fungal infection is caused by fungi selected from Absidia spp., Actinomyces spp., Aspergillus spp., Botrytis spp., Candida spp., Centrospora spp., Cephalosporium spp., Ceratocystis spp., Chaetoconidium spp., Chaetomium spp., Cladosporium spp., Colletotrichum spp, Conidiobolus spp., Fulvia spp., Fusarium spp. (including Fusarium oxysporum), Geotrichum spp., Guignardia spp., Helminthosporium spp., Histoplasma spp., Lecythophora spp., Malassezia spp., Nectria spp., Nocardia spp., Oospora spp., Ophiobolus spp., Paecilomyces spp., Paracoccidioides brasiliensis, Penicillium spp Phymatotrichum spp., Phytophthora spp., Pythium spp., Piedraia hortai, Rhizoctonia spp., Rhizopus spp., Rhodosporidium spp. Saccharomyces spp., Scerotium spp., Sclerotinia spp., Torulopsosis spp., and Trichophyton spp. In a preferred embodiment, the fungal infection is caused by Fusarium spp. In a preferred embodiment, the fungal infection is caused by Fusarium oxysporum.

In some embodiments, the protozoan infection is caused by protozoa selected from the genus Plasmodium, Entamoeba, Giardia, Toxoplasma, Cryptosporidium, Trichomonas, Trypanosoma, Leishmania, Acanthamoeba, Naegleria, Balantidium, Babesia, and Cyclospora. Preferably, the protozoan infection is caused by protozoa of the genus Plasmodium.

In some embodiments, the algal infection is caused by microalgae selected from the genus Prototheca, Helicosporidium, Chlorella and Desmodesmus. Preferably, the algal infection is caused by algae of the genus Prototheca. More preferably, the algal infection is an udder infection.

The determination of acute versus chronic infection is known to the practitioner. For example, according to the Mayo Clinic, the occurrence of a yeast infection 4 or more times within a year indicates the presence of a chronic yeast infection whereas the occurrence of two or more bladder infections during a 6-month period indicates the presence of a chronic bladder infection (also referred to as recurrent urinary tract infection).

The most common method to treat a pathological infection of bacteria is the use of antibiotics. Current antibiotics operate primarily through growth-dependent mechanisms and target rapidly-dividing bacteria. However, non-replicative or slower growing bacteria (e.g., dormant persister cells) display high levels of antibiotic tolerance and/or resistance contributing to persistent and recurring infection. The compounds disclosed herein are suitable for the use in infections comprising antibiotic resistant bacteria, antibiotic tolerant bacteria, and antibiotic persistent bacteria. The compounds disclosed herein are also suitable as a second-line therapy, or rather for individuals that did not response to previous treatment (e.g., antimicrobial treatment) or the disorder returned within e.g., one year or 6 months.

As shown in Example 5, the compounds disclosed herein also lead to little or no resistance. This is an advantage to antibiotic treatment.

In some embodiments, microbial infections also include infections caused by microbes attached on indwelling devices (e.g., medical implants, catheters, etc.). In some embodiments, the compounds and compositions as disclosed herein are useful for treating and preventing infections of implanted medical devices such as joint prosthesis and heart valves as disclosed further herein.

In some embodiments, the compounds and compositions as disclosed herein are also useful for preventing or reducing inflammation in response to infection. Inflammation is part of the complex biological response of body tissues to harmful stimuli, such as pathogens, and is a protective response involving immune cells and molecular mediators. A function of inflammation is to eliminate the pathogens.

In a preferred embodiment, treatment of an individual with the compounds as disclosed herein or compositions comprising same prevents or reduces a clinical inflammation in an animal, e.g., a cow. Preferably the treatment prevents or reduces a (clinical) inflammation of the udder. In another embodiment, treatment of an individual with the compounds and compositions comprising same prevents or reduces a (clinical) inflammation in a human. For example, the treatment prevents or reduces inflammation of the skin, preferably prevents eczema.

In addition to the compounds described herein, further anti-inflammatory drugs can be administered to suppress the inflammatory response and reduce the tissue damage. In a preferred embodiment, the treatments disclosed herein (both therapeutic and prophylactic) further comprise the administration of an anti-inflammatory agent. Anti-inflammatory agents include, for example, nonsteroidal anti-inflammatory agents (cox/lox inhibitors) such as ibuprofen, paracetamol, aspirin, diclofenac, ketoprofen, tolmetin, etodolac, and fenoprofen. Natural anti-inflammatory agents such as Curcumin, Ginger, Spirulina, Cayenne, Cinnamon, Clove, Sage, Rosemary, Black Pepper, natural aspirins, Boswelia, Sanguinaria, and/or Green Tea may also be used. In some embodiments, the methods and uses disclosed herein comprise the combined treatment of the therapeutic organosulfur compounds disclosed herein with an anti-inflammatory agent. The compounds may be administered together or separately. In some embodiments, compositions are provided comprising the therapeutic organosulfur compounds disclosed herein with an anti-inflammatory agent.

In some embodiments, the methods comprise administering to an individual in need thereof compositions comprising the compounds disclosed herein, preferably such as to treat or prevent infection (in particular bacterial, fungal, yeast, protozoan, or algal infection). In some embodiments, the composition can be administered to an individual for the treatment (e.g., therapeutic agent) or prevention (e.g., prophylactic agent) of a disease or disorder or infection. In some embodiments the individual has or is at risk of developing a microbial infection.

The compositions can be administered to any individual, in particular to animals. Preferable, the animal is a ruminant (such as cows and goats), more preferably a cow. In some embodiments, the animal is not a cow. Preferably, the animal is a non-ruminant, such as a monogastric, a rodent, non-human primate, porcine, equine, canine, feline, or avian. In preferred embodiments the animal is a human. In some embodiments the animal is a non-human animal. In some embodiments, the animal is an aquatic animal such as fish, mollusks, and crustaceans. Preferably, the animal is a mammal or bird.

While not wishing to be bound by theory, the disclosure provides that the compositions disclosed herein can have advantageous effects after a single administration. In a preferred embodiment, effects are achieved by providing a single oral administration of the composition disclosed herein. Such oral dosing may be, e.g., as a tablet which provides an extended release of the compounds disclosed herein.

The disclosure also provides for multiple administrations. For example, the compositions may be provided more than once per day, daily, weekly, or monthly. In an exemplary embodiment the composition may be provided once daily for a week or until symptoms are alleviated. Since the compounds disclosed herein lead to little to no resistance, they may be discontinued once symptoms are alleviated or used for multiple treatments, if needed.

Actual dosage levels of the pharmaceutical preparations described herein may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

A skilled person is aware that as smaller animals have higher metabolic rates and thus smaller animals require a larger drug dose on weight basis. Dose conversions between animals, and between humans and animals, are reviewed in Nair and Jacob (J Basic Clin Pharm. March 2016-May 2016; 7 (2): 27-31) and Holliday, et al., (1967 The Relation of Metabolic Rate to Body Weight and Organ Size. A Review. Pediat. Res. 1:185-195).

In some embodiments of the methods and uses disclosed herein, at least 5 mg/day of compounds disclosed herein are provided to a human (such as by oral administration). Preferably, at least 10 mg/day of the compounds are provided. In some embodiments, a compound as disclosed herein is provided to a human at a dose of between 0.1 mg/kg to 100 mg/kg. Such amounts of the compounds are particularly useful when providing the compounds systemically (e.g., orally). A skilled person will recognize that lower amounts can be used when administered locally (e.g., on the skin, gums, wound). The compositions disclosed herein are preferably provided for at least one week or until symptoms are alleviated. While such compositions may be provided several times (e.g., one a week, once a month, twice a year, etc.), prophylactic and therapeutic effects are observed after a single use.

In some embodiments, a composition is provided comprising the compounds as disclosed herein together with one or more additional agents, such as, antibiotics (e.g., antibacterial agents, antiviral agents, anti-fungal agents), anti-inflammatory agents, anti-pyretic agents, and pain-relieving agents.

In some embodiments, the compounds disclosed herein are used together with another antimicrobial agent such as antifungal drugs or antibiotics. As a skilled person will appreciate, the combination of an antimicrobial with the compounds described herein can reduce the dosage and/or dosage frequency of the antimicrobial. Exemplary antimicrobials which may be used in the combination treatment include antifungals such as miconazole, ketoconazole, econazole, terbinafine, ciclopirox, tolnaftate, sertaconazole, sulconazole, amphotericin b, cholorxylenol, clioquinol, butenafine, naftifine, nystatin, and clotrimazole. Exemplary antibiotics include Penicillins, Tetracyclines, Cephalosporins, Quinolones, Lincomycins, Macrolides, Sulfonamides, Glycopeptides, Aminoglycosides, and Carbapenems. The disclosure provides compositions comprising a compound disclosed herein together with an antimicrobial. As a skilled practitioner will appreciate, the compound and an antimicrobial may also be provided separately. In some embodiments the compound and an antimicrobial therapy overlap. In some embodiments, the therapy with a compound of the invention precedes antimicrobial therapy.

In some embodiments, the compositions disclosed herein are provided as a foodstuff composition or in a food product or a functional food product.

The term “foodstuff” as used herein, refers to any liquid or solid substance intended for consumption to provide nutritional support and energy to a subject/an organism. Foodstuff include any type of food or animal feed, as well as functional foods. The foodstuff composition can for, example, be mixed into the foodstuff or can be applied to the surface of the foodstuff (e.g. as a spray or liquid form). The foodstuff may also be immersed in said foodstuff composition. In some embodiments, the foodstuff composition prevents or inhibits spoilage of a foodstuff by microbes. In some embodiments, the foodstuff composition inhibits the growth of microbes and/or kills microbes on the foodstuff. A skilled person is able to identify a suitable concentration to obtain a desired growth inhibition or killing of the microbes.

The term “functional food” as used herein, refers to those foods that are prepared not only for their nutritional characteristics, but also to fulfil a specific function, such as improving health or reducing the risk of contracting diseases. Such functional foods may also be referred to as dietary supplements or (animal) food additive. To this end, biologically active compounds, such as minerals, vitamins, fatty acids, bacteria with beneficial effects, dietary fibre and antioxidants, etc., are added thereto. Such food products may be in any form suitable for oral consumption, e.g., in the form of a liquid, gel, powder, pill, tablet, or in gel capsules.

The functional food may also include animal digest, e.g., any material that results from chemical and/or enzymatic hydrolysis of clean and undecomposed animal tissue.

The functional food may also include dried brewers yeast, e.g., the dried, inactive agent that is a byproduct of the brewing industry. The animal digest and dried brewers yeast have been found to enhance the palatability of the functional food. When present in the functional food, the animal digest comprises from about 10% to about 90% of the functional food and the dried brewers yeast comprises from about 1% to about 30% of the functional food.

In some embodiments, the disclosure provides compositions comprising the compounds of the invention together with at least one pharmaceutically acceptable carrier, diluent and/or excipient. (See e.g., Remington: The Science and Practice of Pharmacy, Alfonso R. Gennaro (Editor) Mack Publishing Company, April 1997). As used herein, the term “pharmaceutically acceptable” refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio. Furthermore, the term “pharmaceutically acceptable diluent or carrier” refers to a pharmaceutically acceptable material, composition or vehicle, such as a liquid or solid filler, diluent, excipient, solvent or encapsulating material, involved in carrying or transporting the peptide from one organ, or portion of the body, to another organ, or portion of the body.

The pharmaceutical composition may be administered by any suitable route and mode. As will be appreciated by the person skilled in the art, the route and/or mode of administration will vary depending upon the desired results. The pharmaceutical compositions may be formulated in accordance with routine procedures for administration by any routes, such as parenteral, topical (including ocular), oral, sublingual, transdermal, or by inhalation. Parenteral administration includes, e.g., intravenous, intramuscular, intra-arterial, intracoronary, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Preferred routes are oral or topical administration.

The compositions may be in any suitable forms, such as liquid, semi-solid and solid dosage forms. The compositions may be in the form of tablets, capsules, powders, granules, lozenges, creams or liquid preparations (in particular for administration to the skin or eye), such as sterile parenteral solutions or suspensions or in the form of a spray, aerosol or other conventional method for inhalation. The pharmaceutical compositions of the present invention include those suitable for oral, nasal, topical (including buccal and sublingual), rectal, vaginal and/or parenteral administration. In particular embodiments, the composition is a topical composition in the form of a cream, gel, ointment, lotion, foam, suspension, spray, aerosol, or powder aerosol. The compositions are particularly useful for administration to the skin. Suitable compositions also include oral care compositions, e.g., toothpaste, dentifrice, tooth powder, tooth gel, subgingival gel, mouthrinse/mouthwash, artificial saliva, denture product, mouthspray, lozenge, oral tablet, and chewing gum.

The disclosure also provides a cleaning or disinfecting product comprising one or more compounds disclosed herein or compositions disclosed herein. In some embodiments, said product is a solution. The solutions include cleaning solutions, sanitizing solutions and disinfecting solutions. In some embodiments, said product is an article, such as wipe, cloth, pad or sponge.

The invention also provides cleaning, sanitizing or disinfecting compositions comprising the compounds of the invention. Preferably, said compositions further comprise at least one surfactant. Suitable surfactants include anionic surfactants, cationic surfactants, nonionic surfactants or amphoteric surfactants. In some embodiments, the cleaning or disinfecting compositions comprise anionic surfactants. Anionic surfactants include, for example, alkyl sulfates (e.g., sodium lauryl sulfate, sodium laureth sulfate, ammonium lauryl sulfate, ammonium laureth sulfate, sodium myreth sulfate), sulfonates (e.g., perfluorooctanesulfonate, perfluorobutanesulfonate), alkyl ether phosphates, alkyl-aryl ether phosphates, carboxylates (e.g., sodium stearate, perfluorooctanoate).

In some embodiments, the cleaning or disinfecting compositions comprise cationic surfactants. Suitable cationic surfactants include, for example, quaternary ammonium compounds and salts thereof (e.g., cetrimonium bromide, benzalkonium chloride, benzethonium chloride, dimethyldioctadecylammonium chloride, dioctadecyldimethylammonium bromide, cetylpyridinium chloride).

In some embodiments, the cleaning or disinfecting compositions comprise nonionic surfactants. Suitable nonionic surfactants include, for example, fatty alcohol ethoxylates, alkylphenol ethoxylates, ethoxylated amines, fatty acid amides (e.g., cocamide monoethanolamine, cocamide diethanolamine), poloxamers, polyethylene glycols, fatty acid esters of glycerol (e.g., glycerol monostearate, glycerol monolaurate), alkyl polyglucosides (e.g., decyl glucoside, lauryl glucoside, octyl glucoside), sorbitan esters (e.g., sorbitan monolaurate, sorbitan monostearate, sorbitan tristearate), polysorbates (e.g., Tween 20, Tween 80).

Suitable amphoteric surfactants include alkylamidopropylamine oxides, alkyldimethylamine oxides, betaines (e.g., lauryl betaine, cocoamidopropyl betaine), lauryldimethylamine oxide, myristamine oxide

In some embodiments, the compositions comprise at least 0.1% wt of surfactant.

The disclosure also provides in vitro methods comprising applying the compositions as disclosed herein to a surface. Preferably, the method is a method of cleaning, sanitizing or disinfecting. More preferably, the method is a method of sanitizing or disinfecting. The compositions used in said methods include, e.g., cleaning compositions, sanitizing compositions, disinfecting compositions. In some embodiments, the method comprises contacting microbes adhered to a surface with the compositions disclosed herein.

Any surface may be treated with the compounds disclosed herein or the compositions disclosed herein so as to coat such surfaces. The surfaces may be, e.g., sprayed, dipped, wiped or soaked in the compositions. A surface includes glass, metal, porous, and non-porous surfaces. It also pertains to exterior and interior and surfaces of equipment that can be contaminated, such as those found in the food industry or the medical equipment found in hospitals and health care facilities, as well as plumbing systems (e.g., sink drain), countertops, building materials, ductwork, clean rooms. A surface also refers to the interior or exterior of pipes, for example drains, as well as swimming pools, tanks (e.g., for aquaculture), purification filters, toilet bowl, sinks, surfaces in the greenhouse. A surface also includes water, such as from a drinking trough.

In some embodiments the surface is of a medical device, such as prosthetics (hip implants, dental implants, prosthetic joint, a voice prosthetic, a penile prosthetic) a mechanical heart valve, a cardiac pacemaker, an arteriovenous shunt, a schleral buckle, catheters (e.g., central venous catheter, an intravascular catheter, an urinary catheter, a Hickman catheter, a peritoneal dialysis catheter, an endrotracheal catheter), tympanostomy tube, a tracheostomy tube, a surgical suture, a bone anchor, a bone screw, an intraocular lens, a contact lens, an intrauterine device, an aortofemoral graft, or a vascular graft. Other medical devices include those from abdominal drains, biliary tract stents, breast implants, cardiac pacemakers, cerebrospinal fluid shunts, contact lenses, defibrillators, dentures, electrical dialyzers, endotracheal tubes, indwelling urinary catheters, intrauterine devices, intravenous catheters, joint prostheses, mechanical heart valves, nephrostomy tubes, orthopedic implants, peritoneal dialysis catheters, prosthetic heart valves, prosthetic joints allosplastic orthopedic devices, tissue fillers, urethral stents, vascular prostheses, ventilator-associated pneumonia, ventricular assist devices, ventricular derivations, ventricular shunts, and voice prostheses.

In some embodiments the surface is of a surgical device, such as clamp, forceps, scissor, skin hook, tubing, needle, retractor, scaler, drill, chisel, rasp, or saw.

The disclosure also provides an article having a surface at least partially coated with the compounds of the invention or the composition disclosed herein. In some embodiments, the article is a medical device or surgical device.

In some embodiments, the in vitro method is a method of cleaning said surface. The term “cleaning” as used herein, refers to the removal of visible soil (e.g., organic and inorganic material), dirt, debris and/or other impurities from surfaces.

In some embodiments, the in vitro method is a method of sanitizing said surface. The term “sanitizing”, “sanitization”, or “to sanitize” as used herein, refer to reduction of the number of microorganisms to levels considered safe according to public health standards or requirements. Sanitization may not necessarily eliminate all the microorganisms on a treated surface. A skilled person is well aware of said standards and requirements.

In some embodiments, the in vitro method is a method of disinfecting said surface. The terms “disinfecting”, “disinfection” or “to disinfect” as used herein, refer to destruction and/or irreversible inactivation of pathogenic and other types of microorganisms, except bacterial spores. The term “inactivating” or “inactivation as used herein, refer to rendering microorganisms unable to grow/replicate. In some embodiments, the method of disinfecting eliminates all pathogenic and other types of microorganisms. In some embodiments, a disinfectant is a chemical sterilant. Said chemical sterilant is a disinfectant applied for prolonged exposure times and may kill spores. In some embodiments, a disinfectant is a high-level disinfectant, intermediate-level disinfectant or a low-level disinfectant. High-level disinfectants kill all microorganisms except large number of bacterial spores. Intermediate-level disinfectants may kill mycobacteria, vegetative bacteria, most viruses, and most fungi but do not necessarily kill bacterial spores. Low-level disinfectants may kill most vegetative bacteria, some fungi, and some viruses. As a skilled person will appreciate, in some embodiments, a method of disinfecting a surface or the use of a disinfecting composition need not destroy or inactivate all microorganisms or all types of microorganisms in order to have a useful effect.

Sanitization and disinfection results in a reduction of the number of a given microorganisms or colony forming units (CFUs). The efficiency of sanitization or disinfection is commonly described with a log reduction. The term “n-log reduction” or “n-logarithmic reduction” as used herein, refers to a percentage of a given microbe reduced/killed/inactivated by a disinfection or sanitization method. A 1-log reduction refers to the reduction of the microorganism of interest by 90% from the original level (i.e. 10 times smaller); a 2-log reduction refers to the reduction of the microorganism of interest by 99% from the original level (i.e. 100 times smaller); a 3-log reduction refers to the reduction of the microorganism of interest by 99.9% (i.e. 1000 times smaller); etc.

In some embodiments, the disinfection provides at least 3-log reduction, preferably at least 5-log reduction, more preferably at least 6-log reduction. In preferred embodiment, the disinfection provides at least 6-log reduction.

In some embodiments, the sanitization provides at least 1-log reduction, at least 2-log reduction, at least 3-log reduction, at least 5-log reduction, at least 6-log reduction. In preferred embodiment, the sanitization provides at least 3-log reduction.

In some embodiments, the cleaning or disinfecting composition is contacted with the treated surface for a sufficient minimum contact time. In some embodiments, the cleaning or disinfecting composition is in contact with the treated surface for at least 30 seconds, at least 1 minute, at least 5 minutes, at least 10 minutes, at least 15 minutes, at least 25 minutes, at least 30 minutes, at least 1 hour, at least 2 hours.

Sanitization and disinfection may provide a residual efficacy or long-lasting efficacy against microorganisms. In some embodiments, the sanitization or disinfection provides residual efficacy for at least 2 hours, at least 6 hours, at least 12 hours, at least 24 hours, at least 48 hours, at least 72 hours, at least a week, at least 2 weeks, at least a month.

In some embodiments, the sanitization or disinfection provides residual efficacy for up to 2 hours, up to 6 hours, up to 12 hours, up to 24 hours, up to 48 hours, up to 72 hours, up to a week, up to 2 weeks, up to a month.

The disclosure also provides uses of the compounds or compositions disclosed herein as a disinfectant, a sanitizing agent, or an antimicrobial agent in foodstuff.

In some embodiments, the use of antimicrobial agent in foodstuff prevents or inhibits spoilage and/or degradation of the foodstuff by microbes. In some embodiments, the use of said antimicrobial agent inhibits the growth of microbes and/or kills microbes on the foodstuff. A skilled person is able to identify a suitable concentration of said antimicrobial agent to obtain a desired growth inhibition or killing of the microbes in foodstuff.

Furthermore, the disclosure provides agricultural composition comprising one or more of the compounds of the invention and an agricultural acceptable excipient and/or carrier. Such carriers and solvents are known to a skilled person and are not unacceptably damaging to a plant or its environment, and/or not unsafe to the user or others that may be exposed. For example, an agriculturally acceptable carrier may be a solid carrier, a gel carrier, a liquid carrier, a suspension, or an emulsion. A non-limiting example of a solvent is water.

In some embodiments, the composition comprises at least 40 wt %, preferably at least 50 wt %, of one or more compounds of the invention. In some embodiments, the composition comprises at least 60 wt %, preferably at least 80 wt %, more preferably at least 95 wt %, of one or more compounds of the invention.

The disclosure includes a method of preventing or treating an infection on a plant or plant part, comprising contacting a plant or plant part with the compounds of the invention or the compositions disclosed herein.

The disclosure includes use of the compounds of the invention or the composition disclosed herein for preventing or treating an infection on a plant or plant part.

The skilled person is capable of establishing whether the compounds of the invention or the composition disclosed herein prevent or treat plant infection. Different bacteria and fungi attack different plants and plant parts and cause different symptoms. For example, bacteria or fungi may cause changes in quality traits, such as changes in color, shape, size, firmness, etc. of the plant or plant part; rotting, wounds; or wilting depending on the plant genus or species. Typically, the skilled person, e.g. a farmer or a grower, knows which plant relates to which quality trait. The compounds or compositions disclosed herein have advantageous effects on plants or plant parts. In particular, the compounds or compositions disclosed herein prevent or treat infections on plant or plant parts. In some embodiments, the compounds or compositions disclosed herein may improve one or more quality traits as compared to infected plants.

In some embodiments, the infection on a plant or plant part is caused by bacteria or fungi. In some embodiments, the infection on a plant or plant part is caused by bacteria selected from one or more Xanthomonas spp., Erwinia amylovora, Rhizobium spp., Clavibacter michiganensis, Agrobacterium radiobacter, Burkholderia spp., Pseudomonadota spp., Pseudomonas spp. (preferably Pseudomonas syringae), Phytoplasma spp. and Spiroplasma spp.

In some embodiments, the infection on a plant or plant part is caused by bacteria selected from one or more Acidovorax, Bacillus, Dickeya, Pectobacterium, Pantoea, Burkholderia, Erwinia, Ralstonia, Rhizobium, Streptomyces, Clavibacter, Xylella, Vitis vinifera and Agrobacterium.

In some embodiments, the infection on a plant or plant part is caused by bacteria selected from one or more Pseudomonas syringae pathovars, Ralstonia solanacearum, Agrobacterium tumefaciens, Xanthomonas oryzae pv. Oryzae, Xanthomonas campestris pathovars, Xanthomonas axonopodis pathovars, Erwinia amylovora, Xylella fastidiosa, Dickeya dadantii, Dickeva solani, Pectobacterium carotovorum and Pectobacterium atrosepticum.

In some embodiments, the infection on a plant or plant part is caused by bacteria selected from one or more Xanthomonas campestris, Erwinia amylovora, Rhizobium spp., Clavibacter michiganensis, and Agrobacterium radiobacter.

In some embodiments, the infection on a plant or plant part is caused by fungi selected from one or more Pythium ultimum, Fusarium oxysporum, Fusarium solani, Phytophthora cactorum, Rhizoctonia spp. (e.g., Rhizoctonia solani), Oidium spp., Uncinula spp., Erysiphe spp., Fusarium spp., Thielaviopsis spp., Verticillium spp., Magnaporthe grisea, Sclerotinia sclerotiorum, Ustilago spp., Phakospora pachyrhizi, Puccinia spp., and Armillaria spp.

In some embodiments, the infection on a plant or plant part is caused by oomycetes, such as, e.g. Pythium spp. and Phytophthora spp. In some embodiments, the infection on a plant or plant part is caused by Phytomyxea spp.

Said compounds or compositions may be applied to a plant or plant part (including cuttings, emerging seedlings, and established vegetation, including roots and above-ground portions, for example, leaves, stalks, flowers, fruits, branches, limbs, root, and the like), to the seed of a plant (e.g., prior to germination), or to the surrounding soil, in particular the plant rhizosphere. As used herein, the term rhizosphere refers to area of soil adjacent to the roots of living plants. The width of the rhizosphere is generally within 100 mm from the root surface.

In some embodiments, the compounds of the invention or compositions comprising same are applied directly to said plant, to the seed of said plant or to the soil of said plant or seed.

As used herein, the term “plant” encompasses crop plants, ornamentals, trees, grasses, annuals, perennials or any other commonly cultivated member of the kingdom Plantae. The term “crop plant” as used herein includes plant species with commercial value, which are planted and cultivated for commercial use. Thus, crop plants include floral and non-floral plants, perennials and annuals, trees, shrubs, vegetable plants, fruit trees, turf, and ground cover.

In some embodiments, the plant belongs to a family selected from the group consisting of begonia family (Begoniaceae), nightshades (Solanaceae), amaranths (Amaranthaceae), roses (Rosaceae), crucifers (Brassicaceae or Cruciferae), orchids (Orchidaceae), composite (Asteraceae or Compositae), Fabaceae, cucumber (Cucurbitaceae), Vitis and Vaccinia.

In some embodiments, the plant belongs to a family selected from the group consisting of begonia family (Begoniaceae), nightshades (Solanaceae), amaranths (Amaranthaceae), roses (Rosaceae) and crucifers (Brassicaceae or Cruciferae).

In a preferred embodiment, the plant belongs to the begonia family (Begoniaceae). Preferably, the plant belongs to the genus Begonia.

In a preferred embodiment, the plant belongs to the nightshades family (Solanaceae). Preferably, the plant belongs to the genus Solanum. Preferably, the plant is selected from the group consisting of tomato (S. lycopersicum), potato (S. tuberosum), eggplant (S. melongena), and pepino (S. muricatum). More preferably, the plant is tomato (S. lycopersicum) or potato (S. tuberosum).

In a preferred embodiment, the plant belongs to the amaranth family (Amaranthaceae). Preferably, the plant belongs to the genus Beta. Preferably, the plant is selected from the group consisting of sugar beet (B. vulgaris, Altissima group), spinach beet or chard (B. vulgari, Cicla group), swiss chard (B. vulgaris, Flavescens group), beetroot (B. vulgaris, Conditiva group), and mangold (B. vulgaris, Crassa group). More preferably, the plant is sugar beet (B. vulgaris, Altissima group).

In a preferred embodiment, the plant belongs to the rose family (Rosaceae). Preferably, the plant belongs to the genus of strawberries (Fragaria) or apples (Malus).

In a preferred embodiment, the plant belongs to the crucifers family (Brassicaceae or Cruciferae). Preferably, the plant belongs to the genus Brassica.

In a preferred embodiment, the plant belongs to the orchid family (Orchidaceae). In exemplary embodiments, the plant is an ornamental plant such as an orchid, in particular of the Phalaenopsis genus, or another flowering plant such as from the genus Cymbidium.

In a preferred embodiment, the plant belongs to the composite family (Asteraceae or Compositae). Preferably, the plant belongs to the genus of Chrysanthemum, Asterea, or Lactuca.

In a preferred embodiment, the plant belongs to the Fabaceae family. Preferably, the plant belongs to the genus of Pisum or Phaseolus.

In a preferred embodiment, the plant belongs to the cucumber family (Cucurbitaceae). Preferably, the plant belongs to the genus Cucumis or Cucurbita. Preferably, the plant belongs to the genus Cucumis and is selected from the group consisting of cucumber (C. sativus), sugar melon, and gherkin (C. anguria). In other preferred embodiments, the plant belongs to the genus Cucurbita and is selected from the group consisting of C. pepo (in particular zucchini), and pumpkins (C. argyrosperma, C. digitate, C. maxima, and C. moschata).

In a preferred embodiment, the plant belongs to the Vitis family.

In a preferred embodiment, the plant belongs to the Vaccinia family.

In a preferred embodiment, the plant is selected from a group consisting of Begonia, Tomato, Potato, Sugar beet, Strawberries, Cabbage, Apples, Orchidaceae, Chrysanthemum, Fabaceae, Cucurbitaceae, Pisum, Vitis, Vaccinia and Lactuca.

In a preferred embodiment, the plant is selected from a group consisting of Begonia, Tomato, Potato, Sugar beet, Strawberries, Cabbage and Apples.

The compounds or compositions disclosed herein may be applied in a single administration or as multiple administrations. For example, the compositions may be provided daily, weekly, monthly or annually. In an exemplary embodiment the compounds or compositions may be provided once daily for a week or until the said compounds or compositions become effective.

In some embodiments, the compositions disclosed herein are provided as a spray solution. When the compositions are sprayed on the plants, the solution may be deposited on the plant parts (e.g. leaves) as droplets with a small surface volume ratio that evaporate, which may lead to the compositions remaining on the plant parts (e.g. leaves) as a residue. This effect can be reduced by including a wetting agent to the compositions.

The amount of compounds or compositions applied will depend upon a variety of factors including, the method of administration, the time of administration, the rate of decomposition of the particular compound being employed, the duration of the treatment, pesticide treatment, compounds and/or materials used in combination, the age, weight, general health and prior treatments, and like factors well known in the agricultural arts. A horticulturist, plant grower or a farmer having ordinary skill in the art can readily determine the effective amount of the compounds or composition required.

It is clear to a skilled person that lower concentrations/amounts of the compounds disclosed herein can be administered to slow growing plants, e.g. cactus and succulents. It is also clear to a skilled person that the concentrations/amounts in water and frequency of application are dependent on the plant species, subspecies, cultivar, hybrid, variant. Furthermore, it is clear to a skilled person in the art that the dosage that the plants can tolerate, is dependent on the growth stage and size of the plant. Furthermore, it is clear to a skilled person in the art that the concentrations/amounts in water and frequency of application are dependent on the growth conditions e.g. light, temperature, evaporation, nutrient concentration and pH in the root substrate, air movement and the application of other pesticides. Furthermore, it is clear to a skilled person in the art that the concentrations/amounts in water and frequency of application is dependent on the moment that it is administered, day, night and the season and weather conditions.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The compounds and compositions disclosed herein are useful as therapy and in therapeutic treatments and may thus be useful as medicaments and used in a method of preparing a medicament. In some embodiments, the disclosure provides methods which are not a treatment of the human or animal body and/or methods that do not comprise a process for modifying the germ line genetic identity of a human being, wherein the cell is not a human germ cell line.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1: Synthesis on 50 grams scale of bis(p-chlorophenyl) thiosulfonate, bis(p-fluorophenyl) thiosulfonate and bis(iso-propyl) thiosulfonate

Scope. In this example the synthesis is described of bis(p-chlorophenyl) thiosulfonate (compound 1a, bis(p-fluorophenyl) thiosulfonate (compound 1b) and bis(iso-propyl) thiosulfonate (compound 2).

Experimental design. The synthesis of the compounds was started with test-runs of 10 g to evaluate the reaction. Based on the obtained yield and observations further scale up was performed or investigated in more detail.

### Synthesis of bis(p-chlorophenyl) thiosulfonate (compound 1a)

The first attempt was performed following the proposal on 10 g scale, using oxidation conditions found in literature (ACS Catal. (2020), 10, p. 8765-8779).

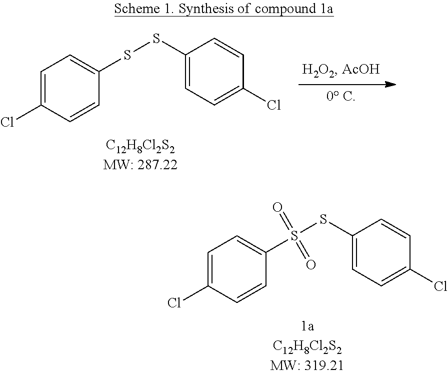 [SMILES: Clc1ccc(SSc2ccc(Cl)cc2)cc1 ; O=S(=O)(Sc1ccc(Cl)cc1)c1ccc(Cl)cc1]

The first test reaction, in which the mixture was kept at 48 h at room temperature, afforded compound 1a as a mixture with the starting material and compounds 3a and 7.

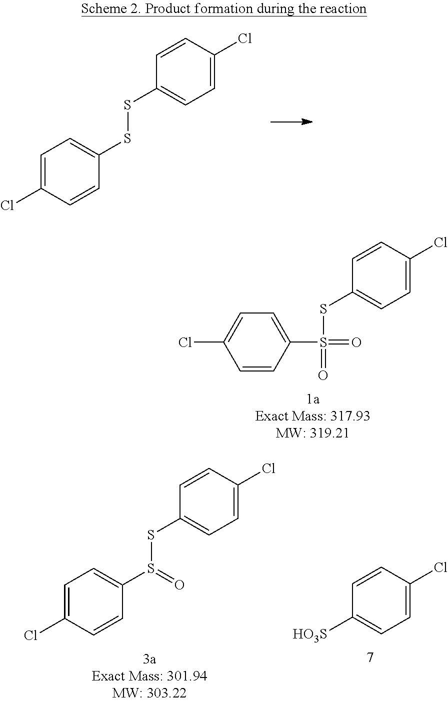 [SMILES: Clc1ccc(SSc2ccc(Cl)cc2)cc1 ; O=S(=O)(Sc1ccc(Cl)cc1)c1ccc(Cl)cc1 ; O=S(Sc1ccc(Cl)cc1)c1ccc(Cl)cc1 ; O=S(=O)(O)c1ccc(Cl)cc1]

Crude compound 1a was analysed by LCMS-24 at UV 265 nm. For analysis an Agilent 1290 Infinity II series with UV detector, ELSD 1290 infinity II detector, and Agilent 6135 mass detector were used equipped with a Waters XSelect CSH C18 2.5 μm 2.1×50 mm (PN: 186006101) column. As a Mobile Phase A: 10 mM Ammonium bicarbonate, pH 9.5 (aq) and as mobile Phase B Acetonitrile was used. UV Detection took place at 265 nm. The main peak was eluted with Rt=3.86 min. Mass spectrometry of the main peak (Rt=3.86 min) showed a mixture of compounds 1a [M+18] and compound 3a.

The crude product has been washed with hot water to remove 4-chlorobenzenesulfonic acid, this treatment removed also other minor impurities and the starting material remained in the mixture. The cake has subsequently been washed with pentanes, which removed the starting material, to afford pure compound.

After the first 10 g test, a second batch has been performed on 50 g scale. After purification, the amount of the two batches did not meet the required amount.

After this, a second synthesis was performed with 50 g substrate. The batches were combined and compound 1a (50 g) was shipped with a purity of 98%. Purity was determined by LCMS-5 CLM (sequence method) (Waters Acquity HSS T3 (2.1×75 mm; 1.8 μm; RRHD 1200 bar, Mobile phase A: 10 mM NH4OAc (Water/Methanol/Acetonitrile 900/60/40; Mobile phase B: 10 mM NH4OAc (Water/Methanol/Acetonitrile 100/540/360.

### Synthesis of bis(p-fluorophenyl) thiosulfonate (compound 1b)

Bis(p-fluorophenyl) thiosulfonate was synthesized by oxidation of 4-fluorobenzenethiol using N-Chlorosuccinimide as described in the scheme below.

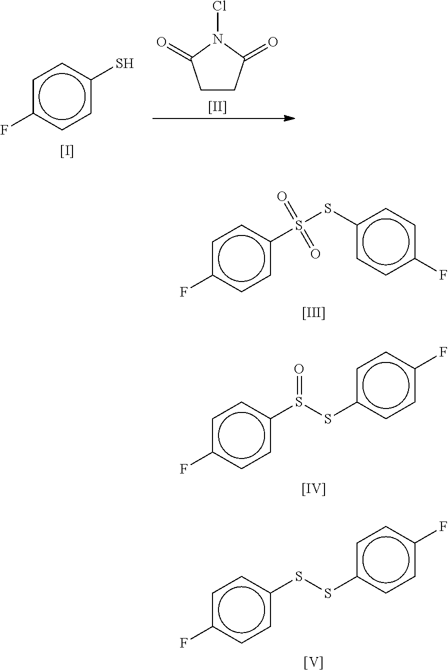

Under air atmosphere at room temperature, 10 mmol (1.28 g) of p-fluorothiophenol (compound [I]) and 15 mmol (4 g) of N-chlorosuccinimide (compound [II]) were put into a round-bottomed flask, and about 20 mL acetonitrile was added, stirred, and reaction took place at room temperature. After half an hour, the reaction was completed. Immediately after the reaction, the mixture comprised (4-fluorophenyl) thiosulfonate (compound 1b or [III] as in the scheme), bis(4-fluorophenyl) thiosulfinate (Compound [II]) and bis(4-fluorophenyl) disulfide (compound [V]). Thereafter the reaction solution was transferred to a 250 mL separatory funnel, extracted with ethyl acetate (30 mL×3), the organic phases of the upper layer were combined, dried, filtered, and distilled to obtain a crude product. The crude product was transferred into the round-bottomed flask and slightly heated, and ethyl acetate was added until the crude product was dissolved completely. Hereafter, after sufficient cooling to separate out the solid, the product was filtered to obtain the product bis(p-fluorophenyl) thiosulfonate. A yield was found of 70% (1.00 g). The product is a white solid with a melting point of 69-70° C.

The product was identified by NMR and met the NMR characteristics for bis(p-fluorophenyl) thiosulfonate: 1H NMR (400 MHz, Chloroform-d) δ 7.58-7.55 (m, 2H), 7.36-7.32 (m, 2H), 7.12-7.08 (m, 2H), 7.06-7.02 (m, 2H).

The molecular weight also corresponded with this compound (determined by GC-MS): M=286 dalton.

### Synthesis of bis(isopropyl) thiosulfonate (compound 2)

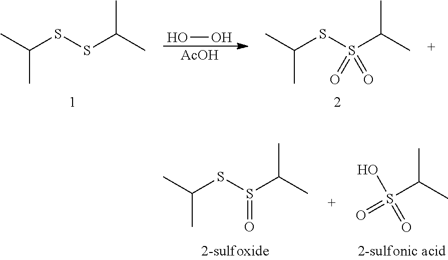

The synthesis of compound 2 was started with 95 g (disulfide 1) scale. First 2.1 mol eq (equivalents: 129 gram) hydrogen peroxide at <45° C. first was added, but the reaction resulted in a mixture of 2 and 2-sulfoxide and potentially also some 2-sulfone (or other side products). After 24 h the reaction was still not complete, and therefore pushed by addition of more hydroxide peroxide (0.5+1.0 eq.); a slight exotherm reaction was observed.

The reaction was followed by NMR (400 MHz, Chloroform-d).

After 48 h at room temperature the reaction was stopped and concentrated under vacuum. Crude material (190.9 g), containing some AcOH and other impurities was directly applied on silicagel (600 g) and eluted in a 0% EtOAc to 20% EtOAc in heptane gradient to result into 37 g of desired compound 2 (bis(4-isopropyl) thiosulfonate) (33% yield).

The reaction was repeated on 50 g scale to obtain the desired amount with a purity of 96.3%. The purity was determined by HPLC-MS. The molecular weight also corresponded with this compound (including NH4 present in the eluent): M=200 dalton.

### Example 2: MIC Values for Some (Potential Pathogenic) Animal Related Microorganisms

### Example 2a: MIC, MBEC and MBIC Assays

The MIC assay in this example measures growth inhibition of planktonic microorganisms. As shown herein, a number of compounds tested show an effect on planktonic bacteria.

TABLE 3
Compounds with CAS number and supplier used in this study.

| Compound | CAS number | Supplier |
|---|---|---|
| Di-n-propyl disulfide | 629-19-6 | Sigma Aldrich |
| Di-isopropyl disulfide | 4253-89-8 | Acros |
| Bis (4-fluorophenyl) disulfide. | 405-31-2 | Fischer Scientific |
| Bis(4-chlorophenyl) disulfide | 1142-19-4 | Thermo Scientific |
| Di-(n-Propyl) thiosulfinate | 1948-52-3 | Organic synthesis |
| Bis(4-chlorophenyl) thiosulfinate | 6481-72-7 | Organic synthesis |
| Bis(4-fluorophenyl) thiosulfinate | 61169-14-0 | Organic synthesis |
| Di (n-Propyl) thiosulfonate | 1113-13-9 | Syncom |
| Di-isopropyl thiosulfonate | 10027-69-7 | Organic synthesis |
| Bis(4-Chlorophenyl) thiosulfonate | 1146-44-7 | Organic synthesis |
| Bis(4-Fluorophenyl) thiosulfonate | 2905-15-9 | Organic synthesis |

### 1.1 Dissolving and Dilution of Compounds

In an Eppendorf tube 20 mg of a compound as indicated in table 3 was weighed. The exact weighed amount was noted and the volume of solvent to reach a 80 mM dilution calculated. Half of the volume was added first, by adding Tween 80 and let stand for 5 minutes. In a second step, the other half of the volume DMSO was added and resuspended to help the dilution process. The closed tube was inverted 5-10 times and then vortexed very thoroughly for at least 30 seconds. If after this procedure crystals were still visible, the tube was placed in a shaker at 37° C. at 150 rpm for at least 30 minutes (it is indicated if the dissolution period was longer than 2 h). The dissolved and transparent dilutions had a concentration of 80 mM and were diluted 10 times with Mueller Hinton Broth 2 cation adjusted (MHB-II) for the MIC assay (method 1.2) to reach the working concentration of 8 mM (0.5 mL needed for one strain). In case of Bis(4-Chlorophenyl) thiosulfonate, a lower concentrated stock solution of 2 mM needed to be prepared. For that, the required amount of compound was weighed in and 2% v/v Tween80 and 2% v/v DMSO of the end volume of the stock solution was added to the substance. This mix was heated in a water bath set to 40° C. and occasionally shaken. When the whole amount of the substance reached dissolution, the volume was filled up with growth medium until the end volume. This 2 mM stock solution of Bis(4-Chlorophenyl) thiosulfonate was used in the MIC plate and diluted two-fold, resulting in a testing range of Bis(4-Chlorophenyl) thiosulfonate from 1 mM to 0.002 mM.

For the MBEC assay (method 1.3) the solutions need to be diluted to a final concentration of 2 mM in 0.9% saline solution and then further diluted to 1 mM, 0.5 mM, 0.25 mM and 0.125 mM in saline. Those dilutions were added as treatment to the 96 well plates with the grown biofilm (see method 3) (0.5 mL needed for one strain). The dilution must stay transparent.

TABLE 4
List of microorganisms used in this study.

| Test Organism | Strain number |
|---|---|
| Staphylococcus epidermidis | ATCC 35984 |
| Pseudomonas aeruginosa | ATCC 27853 |
| Escherichia coli | ATCC 25922 |
| Klebsiella pneumoniae | ATCC 13883 |
| Streptococcus agalactiae | ATCC 13813 |
| Lactococcus garvie | Isolate 2 |
| Streptococcus uberis | ATCC 27985 |
| Streptococcus Suis Type 2 | 21-018358-002 |
| Staphylococcus aureus (MRSA) | LUH14616 |
| Acinetobacter baumanni | RUH875 |
| Micrococcus luteus | ATCC 4698 |

* Kindly supplied by Leids Universitair Medisch Centrum (LUMC), Albinusdreef 2, 2333 ZA Leiden, The Netherlands

### 1.2 MIC Assays

The microorganisms as indicated in Table 4 were used to inoculate a tryptic soy agar (TSA) plate and incubated overnight at 37° C. After incubation 3-5 well-isolated colonies with the same morphology were selected from the TSA plate and resuspended in 2 mL 0.9% saline solution with 3-6 glass beads and vortexed. The optical density at 600 nm (OD₆₀₀) of the bacterial suspension was measured with a spectrophotometer (Evolution™ 201/220 UV-Visible Spectrophotometer, Thermo Fisher Scientific) and diluted to an OD₆₀₀ of 0.0008, which resembles 10⁶ CFU/mL.

The MIC assay was performed in a flat-bottom 96-microtiter plate. 50 μL of MHB-II was added into columns 2-11, and 100 μL to column 12 for sterility control. 100 μL of the 8 mM test compound solution (method 1) was added to column 1. A two-fold dilution series of the solution in MHB-II was achieved by resuspending 50 μL of the 8 mM solution from column 1 to column 2. This step was repeated until column 10. The respective concentration of Tween 80 and DMSO in column 1 was 2.5%, which is two-fold diluted throughout the plate. 50 μL of the bacterial suspension was added to column 1-11. The microtiter plates were sealed with adhesive polyethylene film for sealing microplates (Diversified Biotech) and incubated for 24 h at 37° C. After incubation the OD was measured at 600 nm with the Varioskan (Thermo Fisher Scientific). The minimal concentration which causes reduction of final OD₆₀₀ of the bacterial culture by 50% (MIC50) and by 90% (MIC90) was calculated after normalization of the obtained OD values to the growth control in column 11. This assay was repeated three times in three independent experiments.

### 1.3 MBEC Assays

Frozen aliquots of Staphylococcus epidermis ATCC 35984 and Pseudomonas aeruginosa ATCC 27853 were used to inoculate 20 mL of Tryptic Soy Broth (TSB) in a 100 mL Erlenmeyer flask at 37° C. and 150 rpm and incubated overnight (Infors). The OD₆₀₀ of the overnight culture was measured with a spectrophotometer (Evolution™ 201/220 UV-Visible Spectrophotometer, Thermo Fisher Scientific) and diluted to an ( ) 600 of 0.2, which resembles 10⁸ CFU/mL. The cell suspension of OD_{600=0.2} was seeded into a U-bottom-shaped 96 well plates by pipetting 100 μL per well in column 1-11, column 12 was filled with 100 μL medium as sterility control. The 96 well plates were sealed with adhesive polyethylene film for sealing microplates and incubated for 48 h at 37° C. at static conditions. After the biofilm growth phase, 100 μL of treatment solutions (method 1) were added to the vials resulting in treatment concentrations of 1 mM, 0.5 mM, 0.25 mM and 0.125 mM and with the respective concentration of Tween 80 and DMSO each at 0.63%, 0.32%, 0.16% and 0.08%. The same concentration was applied to four different wells of the same bacterial strain, resulting in four replicates for each concentration and strain. The treatment was incubated for 20 h at 37° C. at static conditions. After the treatment the supernatant was carefully removed and the well with the biofilm washed carefully for one time with 200 μL 0.9% saline solution. 200 μL saline solution was added and the biofilm was resuspended thoroughly. When fully homogenized then 20 μl, was used for the first dilution in 180 μL 0.9% saline solution, which was prepared in a second plate. This dilution was continued until dilution step 10E-5 and 100 μL of the 10E-4 and 10E-5 dilution plated on a TSA plate. The TSA plates were incubated at 37° C. for 24 h. The colonies on the plates were enumerated, the colony forming units per ml, of the dispensed biofilm and averages thereof and the log reduction in comparison with the untreated sample from the same wells plate calculated. Reduction of colony forming units goes hand in hand with the thickness of the biofilm and thus with the number bacteria that are present in the biofilm. Therefore, a significant reduction of colony forming units in the biofilm was defined at the value of log reduction >1 or more. This assay was repeated two times in two independent experiments)

### 1.4 MBIC Assays

Frozen aliquots of Staphylococcus epidermis ATCC 35984 and Pseudomonas aeruginosa ATCC 27853 were used to inoculate a tryptic soy agar (TSA) plate and were incubated overnight at 37° C. After incubation 3-5 well-isolated colonies with the same morphology were selected from the TSA plate and resuspended in 2 ml 0.9% saline solution with 3-6 glass beads and vortexed. The optical density of the bacterial suspension was measured at 600 nm (OD₆₀₀) with a spectrophotometer (Evolution™ 201/220 UV-Visible Spectrophotometer, Thermo Fisher Scientific) and diluted to an OD₆₀₀ of 0.2. The MBIC assay was performed in a 96-microtiter plate. 50 μL of TSB was added into columns 2-11, and 100 μL to column 12 for sterility control. 100 μL of the 8 mM test compound solution (method 1) was added to column 1. A two-fold dilution series of the solution in TSB was achieved by resuspending 50 μL of the 8 mM solution from column 1 in column 2. This step was repeated until column 10. The respective concentration of Tween 80 and DMSO in column 1 is at 2.5%, which is two-fold diluted throughout the plate. 50 μL of the bacterial suspension was added to column 1-11. The microtiter plates were sealed with adhesive polyethylene film for sealing microplates (Diversified Biotech) and incubated for 48 h at 37° C. After incubation the supernatant is carefully removed and the well with the biofilm washed for one time with 100 μl. 0.9% saline solution. 100 μL. 0.1 M HCl was added and incubated for 1 h at room temperature to fix the biofilm. After incubation the HCl was removed and 100 μL, crystal violet (0.1% v/v in water) was added and incubated for 30 min at room temperature. The unbound crystal violet was removed and the wells washed one time with 100 μL demineralized water. 100 μL 30% acetic acid was added and incubated for 1 h at 37° C. and 150 rpm. The solution was resuspended and transferred to a flat-bottom 96-well plate and the absorbance measured at 540 nm with the spectrophotometer. This assay was repeated two times in two independent experiments.

Results and discussion. In Tables 5 and 6 the results of the tests are presented. In Tables 5 and 6, a number of organosulfur compounds showed low MIC-values and are useful as antimicrobial compounds.

TABLE 5
Results of the MIC- assays. MIC50 en MIC90 were defined as the minimal concentration
which causes reduction of final OD600 of the bacterial culture by 50% (MIC50)
and by 90% (MIC90) compared to a culture without the test compound.

| | | | | | S. | P. | | |
|---|---|---|---|---|---|---|---|---|
| | | | | | epidermidis | aeruginosa | S. | P. |
| | S. epidermidis | | P. aeruginosa | | MBEC | MBEC | epidermidis | aeruginosa |
| | MIC50 | MIC90 | MIC50 | MIC90 | mM with log | mM with log | MBIC90 | MBIC90 |
| Compound | [mM] | [mM] | [mM] | [mM] | reduction ≥1 | reduction ≥1 | [mM] | [mM] |
| Di-n-propyl | >4 | >4 | >4 | >4 | >1 | >4 | 4 | >4 |
| disulfide | | | | | | | | |
| Di-isopropyl | >4 | >4 | >4 | >4 | >1 | >1 | | |
| disulfide | | | | | | | | |
| Bis (4-chlorophenyl) | 0.01 | 0.03 | 0.02 | 0.03 | >1 | >1 | >4 | >4 |
| disulfide | | | | | | | | |
| Bis (4-fluorophenyl) | 0.06 | 0.06 | 0.125 | 0.25 | >1 | >1 | | |
| disulfide | | | | | | | | |
| Bis (4-chlorophenyl) | 0.02 | 0.02 | 0.02 | 0.02 | >1 | >1 | | |
| thiosulfinate | | | | | | | | |
| Bis (4-fluorophenyl) | 0.125 | 0.25 | 1 | 4 | >1 | >1 | | |
| thiosulfinate | | | | | | | | |
| Di-n-propyl | 0.25 | 0.25 | 0.25 | 0.25 | 0.25 | 1 | 2 | 0.5 |
| thiosulfonate | | | | | | | | |
| Di-isopropyl | 2 | 2 | 2 | 2 | >1 | >1 | | |
| thiosulfonate | | | | | | | | |
| Bis (4-chlorophenyl) | 0.03 | 0.06 | 0.25 | 2 | >1 | >1 | | |
| thiosulfonate | | | | | | | | |
| Bis (4-fluorophenyl) | 0.25 | 2 | 2 | 2 | >1 | >1 | | |
| thiosulfonate | | | | | | | | |

TABLE 6
MIC50 [mM] and MIC90 [mM] values of tested compounds with different bacteria. The values were determined
by conducting a MIC assay three independent times (n = 3). If a value is expressed as > or < then the
true value lies supposedly above or below the tested range, respectively. ND = not determined; MIC50 = concentration
[mM] where the growth is reduced to 50% of growth control; MIC90 = concentration [mM] where the growth
is reduced by 90% compared to the growth control. Gram positive strains are indicated with a (+).

| | | Bis (4- | | Bis (4- | | Bis (4- | | Bis (4- | | Bis (4- | |
|---|---|---|---|---|---|---|---|---|---|---|---|
| | | chlorophenyl) | | fluorophenyl) | | fluorophenyl) | | chlorophenyl) | | fluorophenyl) | |
| | | disulfide | | disulfide | | thiosulfinate | | thiosulfonate | | thiosulfonate | |
| | | (n = 3) | | (n = 3) | | (n = 3) | | (n = 3) | | (n = 3) | |
| | Strain | Commercial | | Commercial | | Synthesis | | Synthesis | | Synthesis | |
| | number/ | Cas 1142-19-4 | | Cas 405-31-2 | | Cas 61169-14-0 | | Cas 1146-44-7 | | Cas 2905-15-9 | |
| Strains | isolate | MIC50 | MIC90 | MIC50 | MIC90 | MIC50 | MIC90 | MIC50 | MIC90 | MIC50 | MIC90 |
| Escherichia coli | ATCC | >4 | >4 | 1.25 | >4 | 1.00 | >4 | >1 | >1 | 1.50 | >4 |
| | 25922 | | | | | | | | | | |
| Klebsiella pneumoniae | ATCC | >4 | >4 | >4 | >4 | 2.50 | >4 | >1 | >1 | >4 | >4 |
| | 13883 | | | | | | | | | | |
| Streptococcus agalactiae (+) | ATCC | 0.01 | 0.07 | 0.21 | 0.50 | 1.04 | 1.67 | 0.16 | 0.50 | 1.33 | 1.67 |
| | 13813 | | | | | | | | | | |
| Lactococcus garvie (+) | Isolate 2 | 1.35 | >4 | 0.25 | >4 | 0.13 | 0.83 | 0.09 | 0.50 | 0.22 | 0.83 |
| Streptococcus uberis (+) | ATCC | 0.02 | 0.05 | 0.21 | 0.63 | 0.42 | 1.50 | 0.06 | 0.50 | 0.67 | 1.33 |
| | 27985 | | | | | | | | | | |
| Streptococcus Suis Type 2 (+) | 21-018358-002 | 0.06 | 0.05 | 0.38 | 1.67 | 0.38 | 0.83 | 0.13 | 0.37 | 0.75 | 0.88 |
| Staphylococcus aureus (+) | LUH14616 | <0.008 | 0.19 | <0.008 | 2.33 | 0.28 | 1.77 | 0.06 | 0.75 | <0.008 | 1.27 |
| (MRSA) | | | | | | | | | | | |
| Acinetobacter baumanni | RUH875 | >4 | >4 | 3.00 | >4 | 2.00 | >4 | >1 | >1 | 2.83 | >4 |
| Micrococcus luteus (+) | ATCC | 0.05 | 0.25 | 0.75 | 2.00 | 2.00 | 3.33 | 0.21 | 0.75 | 1.50 | 2.00 |
| | 4698 | | | | | | | | | | |
| Staphylococcus epidermidis (+) | ATCC | 0.01 | 0.03 | 0.06 | 0.06 | 0.13 | 0.25 | 0.03 | 0.06 | 0.25 | 2.00 |
| | 35984 | | | | | | | | | | |
| Pseudomonas aeruginosa | ATCC | 0.02 | 0.03 | 0.13 | 0.25 | 1.00 | 4.00 | 0.25 | 2.00 | 2.00 | 2.00 |
| | 27853 | | | | | | | | | | |

### Example 2b: MIC50, MIC90 and MBIC on Animal and Human Pathogens

From example 2a a number of compounds were selected for further antimicrobial studies. Their selection was based on the antimicrobial effect and stability after synthesis. The selected compounds were bis(4-chlorophenyl) disulfide, bis(4-fluorophenyl) disulfide, bis(4-chlorophenyl) thiosulfonate and diisopropyl thiosulfonate and were tested on various suspected human, animal and plant pathogens, bacteria as well as fungi.

Scope. In this study, the microbial growth-inhibiting activities of organo-sulfur compounds were measured. Some of these compounds are commercially obtained (bis(4-chlorophenyl) disulfide and bis(4-fluorophenyl) disulfide). The other compounds were synthesized on request (Table 7; organic synthesis). The measured microbial growth-inhibiting (MIC) and bactericidal (MBC) capabilities were compared with the values measured for the biofilm-eradicating compound di-n-propyl thiosulfonate.

Study design. In this study the microbial growth-inhibiting activities of some organically synthesized (Table 7; organic synthesis) and commercially obtained organo-sulfur compounds were measured.

Since the compounds have a low solubility in water, it was first assessed at which concentration the compounds could be dissolved with the aid of solvents at a concentration that is suitable for biological assays. Hereafter, the stock solutions of the compounds were used in three repetitions in a minimal inhibitory concentration (MIC) assay at a 2-fold dilution range with various bacteria from the gram-positive and gram-negative group and a yeast, Candida albicans. The concentrations that show only less than 10% growth after 24 h were considered the MIC90-values, whereas the concentrations that show 50% growth after 24 h when compared to the bacterial growth control were considered the MIC50-values. Escherichia coli and Staphylococcus aureus were used as representatives for a gram-negative bacteria and gram-positive bacteria, respectively. Furthermore, the yeast C. albicans was also tested. After the MIC assays with diisopropyl thiosulfonate and di-n-propyl thiosulfonate the minimal bactericidal concentrations were determined to investigate whether growth inhibition (bacteriostatic effect) also meant that the cells are killed (bactericidal).

TABLE 7
Compounds with CAS number and supplier used in this study.

| Class | Compound | Cas | Supplier |
|---|---|---|---|
| Di-alkyl | Bis (4-chlorophenyl) disulfide | 1142-19-4 | Apollo |
| disulfides | Bis (4-fluorophenyl) disulfide | 405-31-2 | Thermo- |
| | | | scientific |
| Di-alkyl | Diisopropyl thiosulfonate | 10027-69-7 | Symeres |
| thiosulfonates | Bis (4-chlorophenyl) | 1146-44-7 | Symeres |
| | thiosulfonate | | |
| | Di-n-propyl thiosulfonate | 1113-13-9 | Symeres |

In table 8 the strains and origin of strains are presented.

TABLE 8
List of used strains, strain characteristics and origin.

| | | Gram | |
|---|---|---|---|
| | Strain | positive/ | |
| Strain | number | negative | Strain supplier |
| Escherichia coli | ATCC 25922 | − | LGC Standards |
| Klebsiella pneumoniae | ATCC 13883 | − | LGC Standards |
| Streptococcus | ATCC 13813 | + | LGC Standards |
| agalactiae | | | |
| Lactococcus garvieae | Isolate 2 | + | Isolate from |
| | | | the field |
| Streptococcus uberis | ATCC 29785 | + | LGC Standards |
| Streptococcus Suis | 21-018358- | + | Isolate from |
| (Type 2) | 002 | | the field |
| Staphylococcus aureus | LUH 14616 | + | LUMC |
| (MRSA) | | | |
| Acinetobacter | RUH875 | − | LUMC |
| baumanni | | | |
| Micrococcus luteus | ATCC 4698 | + | LGC Standards |
| Staphylococcus | ATCC 35984 | + | LGC Standards |
| epidermidis | | | |
| Pseudomonas | ATCC 27853 | − | LGC Standards |
| aeruginosa | | | |
| Moritella viscosa | Isolate | − | Norwegian |
| | 12064 | | Veterinary |
| | | | Institute |
| Candida albicans | Y01-19 | n.a. | Pfizer Inc, |
| | | | Groton, CT, USA |

LUMC: Leiden University Medical Centre, Leiden, The Netherlands
n.a.: not applicable

Emulsions of the test compounds were prepared as presented in table 9. In an Eppendorf tube between 10-20 mg of the test compound was weighed. Hereafter, the required amount of Tween 80 was added, followed by the amount DMSO. After vigorous shaking, the dissolved and transparent dilutions were diluted 10 times with Mueller Hinton Broth (MHB) for the MIC assay. In the case of C. albicans Sabourad broth was used and in the case of M. viscosa TSB with 2% NaCl. The stock solutions were used for the assay and diluted in a 2-fold dilution series.

TABLE 9
Composition of the test solutions, tween 80 and DMSO. A medium
Mueller Hinton Broth (MHB) was used for the MIC-assay.

| | Concentration | | | |
|---|---|---|---|---|
| | in 10-fold | Tween 80 | DMSO | Amount |
| | concentrated | concen- | concen- | of added |
| | stock solution | tration | tration | medium |
| Compound | (mM) | (%) | (%) | (ml) |
| Bis (4-chlorophenyl) | 2 | 1 | 1 | 10 |
| disulfide | | | | |
| Bis (4-fluororophenyl) | 8 | 1 | 1 | 10 |
| disulfide | | | | |
| Diisopropyl | 8 | 1 | 1 | 10 |
| thiosulfonate | | | | |
| Bis (4-chlorophenyl) | 1 | 1 | 1 | 10 |
| thiosulfonate | | | | |
| Di-n-propyl | 8 | 1 | 1 | 10 |
| thiosulfonate | | | | |

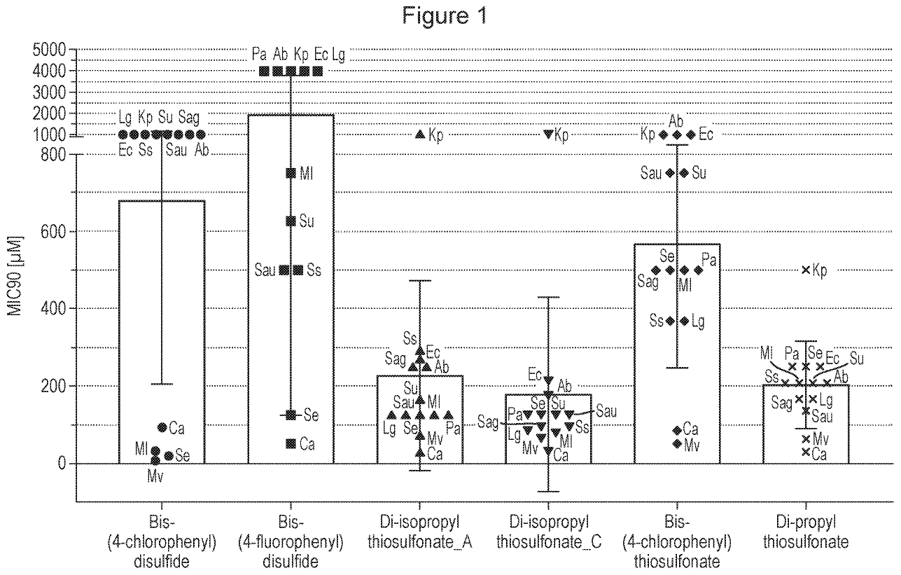

The Minimal Concentration Inhibitory Concentration (MBIC) assay was performed as follows: frozen aliquots of the cells of interest were used to inoculate a tryptic soy agar (TSA) plate and incubated overnight at 37° C. In the case of C. albicans Sabourad agar was used. M. viscosa was inoculated in liquid TSB supplemented with 2% NaCl and incubated for two days in static conditions at 15° C. After incubation 3-5 well-isolated colonies with the same morphology were selected from the plates and resuspended in 2 mL 0.9% saline solution with 3-6 glass beads and vortexed. In the case of M. viscosa the pre-culture was diluted 10 times in sterile demi water. The optical densities at 600 nm (OD₆₀₀) of the bacterial suspension were measured and diluted to an OD₆₀₀ of 0.0008, which resembles 10⁶ CFU/mL. The MIC assay was performed in a flat-bottom 96-microtiter plate. 50 μL of the appropriate growth medium, Sabourad broth for C. albicans, TSB 2% NaCl for M. riscosa, and MHB for the rest of the strains was added to columns 2-11, and 100 μL to column 12 for sterility control. 100 μL of the 8 mM test compound solution (method 1) was added to column 1. A two-fold dilution series of the solution in growth medium was achieved by resuspending 50 μL of the stock solution from column 1 to column 2. This step was repeated until column 10. The respective concentrations of Tween 80 and DMSO in column 1 was half of the concentration in the stock solution, and further two-fold diluted throughout the plate. 50 μL of the cell suspension was added to columns 1-11. The microtiter plates were sealed with adhesive polyethylene film for sealing microplates (Diversified Biotech) and incubated for 24 h at 37° C. After incubation, the OD was measured at 600 nm with the Varioskan (Thermo Fisher Scientific). The minimal concentration that causes a reduction of the final OD₆₀₀ of the culture by 50% (MIC50) and 90% (MIC90) was calculated after normalizing the obtained OD values to the growth control in column 11. This assay was repeated three times in three independent experiments. The MIC90 and MIC50 values were averaged and the standard deviation was calculated. Hereafter, the averages and standard deviations were calculated per strain and compound in a bar graph to compare the antimicrobial activity of the compounds on the different strains (FIG. 1).

The values of the technical replicates in the plates (four per experiment) were plotted over the used concentration in GraphPad to provide information on the scattering of the replicates (results are not shown here).

Conclusion and discussion. The results of the MIC assays (FIG. 1) show that most of the tested compounds have a growth-inhibiting effect on the strains in this study. Bis(4-chlorophenyl) disulfide showed a low solubility. With the addition of solvents like Tween80 and DMSO a stock solution in medium of 2 mM was feasible. This allowed a maximum of testing concentration of 1 mM. Similarly, for bis-(4-chlorophenyl) thiosulfonate the calculated solubility is 0.013 mM. Therefore, with the application of solvents a maximum testing concentration of 0.575 mM was possible. If the real growth-inhibiting concentration (MIC90) lies above these maximum assay concentrations, then it would not be detected in this assay. That was observed for bis(4-chlorophenyl) disulfide with E. coli, K. pneumoniae, S. agalactiae, L. garvieae, S. uberis, S. suis Type 2, S. aureus (MRSA) and A. baumanni and for bis-(4-chlorophenyl) thiosulfonate for E. coli, K. pneumoniae, A. baumanni. For di-n-propyl thiosulfonate and diisopropyl thiosulfonate the solubilities are 5.6 mM and 9.5 mM respectively and therefore, the assay to 4 mM is still in the theoretical solubility.

Furthermore, there was a low batch-to-batch variability for diisopropyl thiosulfonate that showed MIC90 values in a similar range for the same species (FIG. 1, columns Di-isopropyl thiosulfonate A and C for batch A and C, respectively). There was a clear difference in the sensitivity of C. albicans compared to other species tested. C. albicans seems to have a higher sensitivity, hence lower MIC90 values. It is concluded that all compounds show growth-inhibiting effect, dependent on the microorganism.

The Minimal Bactericide Concentration (MBC) of two compounds were determined, namely diisopropyl thiosulfonate and di-n-propyl thiosulfonate. The MBC is the lowest concentration of the compound that did not show any colonies on an agar plate inoculated from the MIC assay and incubated for 20 h at optimal growth temperature depending on the strains. This assay was performed per strain of E. coli, S. aureus, and C. albicans three independent times. Therefore, the standard deviation could also be calculated (Table 10).

TABLE 10
MBC [uM] values of diisopropyl thiosulfonate and di-n-
propyl thiosulfonate for E. coli, S. aureus and C. albicans.
The values were determined by conducting an MBC assay three
independent times (n = 3). The values are expressed with
the standard deviation from the different experiments.

| | Strain | diisopropyl thiosulfonate | | di-n-propyl |
|---|---|---|---|---|
| Strain | number | Batch A | Batch C | thiosulfonate |
| Escherichia coli | ATCC | 219 | 219 | 208 |
| | 25922 | | | |
| Staphylococcus | LUH | 1500 | 1833 | 1083 |
| aureus | 14616 | | | |
| Candida | Y01-19 | 75 | 50 | 25 |
| albicans | | | | |

Conclusion and discussion. It could be observed that di-n-propyl thiosulfonate has the lowest MBC values for all species, and hence has the highest bactericidal activity. The MIC assay (FIG. 1) gives information on growth inhibition in a time window of 24 h in the presence of the compound. If the exposure to the compound is stopped it might be that the (surviving) cells restart growing. By conducting an MBC assay the bactericidal effect of a compound on a particular strain is determined. Di-n-propyl thiosulfonate and diisopropyl thiosulfonate show at concentrations of growth inhibition (MIC90) also a bactericidal effect (MBC) for E. coli and C. albicans. For S. aureus both compounds resulted in higher MBC values compared to MIC90 values, thereby showing a bacteriostatic effect. That means that at concentrations of the MIC90, there were still viable cells present. The bactericidal effect (killing of the pathogens) occurs with an increase of concentration.

### Example 3

Eradication of Staphylococcus aureus LUH 14616 Biofilms by AM-8

Aside from antimicrobial resistant (AMR) bacteria, biofilm contributes to the impaired efficacy of antibiotics against clinical infections (Sharma D, Misba L, Khan A U. Antibiotics versus biofilm: an emerging battleground in microbial communities. Antimicrob Resis Infect Control 8; 76, 2019). A biofilm is a community of bacteria that reside within a (self-produced) matrix of extracellular polymeric substances that protects the bacteria from amongst others the action of antibiotics (review Penesyan A, Paulsen I T, Kjelleberg S, Gillings M R. Three faces of biofilms: a microbial lifestyle, a nascent multicellular organism, and an incubator for diversity. NPJ Biofilms Microbiomes. 2021 7 (1): 80. Doi: 10.1038/s41522-021-00251-2).

Scope. The goal of this in vitro experiment was to assess the efficacy of AM-8 (diisopropyl thiosulfonate) against AMR Staphylococcus aureus in clinical relevant biofilms.

### Experimental Design.

Biofilm eradication assay. The assay to reduce bacterial counts in mature biofilms was assessed as described earlier (Scheper H J, Wubbolts J M, Verhagen J A M, de Visser A W, van der Wal R J P, Visser L G, de Boer M J J G, and Nibbering P H. SAAP-148 eradicates MRSA persisters within mature biofilm models simulating prosthetic joint infection. Front. Microbiol. 12:625952. Doi: 10.3389/fmicb.2021.625952. eCollection 2021) with a single modification, i.e., the biofilms were exposed daily to AM-8 for up to 4 consecutive days. Briefly, approximately 1×10⁷ CFU logarithmic phase bacteria in BHI were cultured in a 96-well flat-bottom polystyrene microplate sealed with breathable seal for 7 days. Next, mature biofilms were washed twice with PBS, then exposed for 24 hrs to AM-8 or as control the diluent of this compound. Hereafter the biofilms were washed and either re-exposed to AM-8/its diluent for another day or—in case of termination of the daily exposures-sonicated for microbiological quantification of the number of viable bacteria. Results are expressed as the number of surviving bacteria at the various days of exposure. Experiment was performed in triplicate.

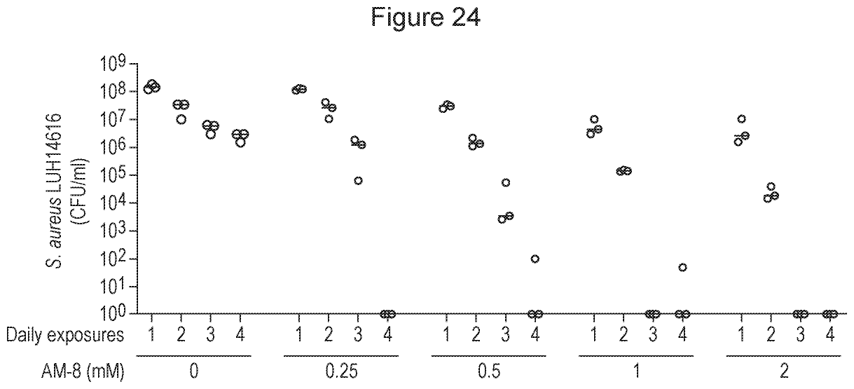

Results. As mature biofilms mimic clinical infections better than 24 hrs immature biofilms (Cazander G, van de Veerdonk M C, Vandenbroucke-Grauls C M, Schreurs M W, Jukema G N. Maggot excretions inhibit biofilm formation on biomaterials. Clin Orthop Relat Res; 468 (10): 2789-96, 2010) and antibiotics poorly affect the bacteria in these bacterial communities, the ability of AM-8 to eradicate S. aureus LUH 14616 in 7 days mature biofilms was assessed. Results of a dose range study revealed that the biofilm reducing effect of a single dose of AM-8 increased in time without fully eradicating the bacteria within the biofilm (results not shown). Therefore, the possibility that multiple daily exposures to AM-8 are more effective against S. aureus LUH 14616 biofilms than a single dose was investigated. Results revealed that with increasing number of daily exposures AM-8 dose-dependently significantly reduced the bacterial counts in mature biofilms (FIG. 24). Together, multiple daily applications of lower doses of AM-8 were more effective than a single exposure to higher doses of this compound.

Conclusion. AM-8 is able to eliminate all bacteria within biofilms, which is almost impossible with antibiotics (see also example 4).

### Example 4: Anti-Persister Experiment

Approximately 80% of the clinical infections are biofilm-associated. Biofilms are structured microbial communities embedded in a 3D extracellular matrix (Penesyan et al (2021). Three faces of biofilms: a microbial lifestyle, a nascent multicellular organism, and an incubator for diversity. NPJ Biofilms Microbiomes 7:80). This matrix provides protection to the entire bacterial community against environmental stressors, including antibiotics and effector molecules of the immune system. In addition, bacteria within biofilms are very heterogenous, including tolerogenic bacteria and persisters, and thrive in different local micro-environments. Persisters are bacterial cells that temporarily reside in a slow- or non-growing, reduced metabolic state that arise both stochastically as well as in response to environmental cues, such as antibiotics, and commonly occur in detectable levels in biofilms (Prax M, and Bertram R. (2014). Metabolic aspects of bacterial persisters. In: Frontiers in Cellular and Infection Microbiology (Vol. 4, Issue OCT). Frontiers Research Foundation). Previous studies have reported that this slow- or non-growing, low metabolic state is associated with tolerance, but not resistance towards antibiotics (Martínez J L, and Rojo F. (2011). Metabolic regulation of antibiotic resistance. In FEMS Microbiology Reviews 35 (5), 768-789). Persisters are likely responsible for re-infections in patients (Lewis K. (2010) Persister cells. Annu Rev Microbiol. 64:57-72). It has been reported that bacteria within biofilms are up to 1,000-fold more resistant to multiple antibiotics than their planktonic counterparts (Sharma D, Misba L, Khan AU (2019). Antibiotics versus biofilm: an emerging battleground in microbial communities. Antimicrob Resis Infect Control 8:76).

Scope. In this experiment the sensitivity of persister bacteria is determined for the antimicrobial compound Bis(4-chlorophenyl) disulfide.

Experimental design. The capacity of AM-2 (Bis(4-chlorophenyl) disulfide) and AM-8 to reduce bacterial counts in mature biofilms was assessed as described earlier (Scheper et al (2021) Scheper H, Wubbolts J M, Verhagen J A M, de Visser A W, van der Wal RJJP, Visser L G, de Boer M J G, and Nibbering P H. SAAP-148 eradicates MRSA persisters within mature biofilm models simulating prosthetic joint infection. Front. Microbiol. 12:625952. Doi: 10.3389/fmicb.2021.625952. eCollection 2021) with one modification, i.e., the biofilms were exposed daily to the compounds for up to 4 consecutive days. Briefly, antimicrobial resistant (AMR) Staphylococcus aureus LUH14616 were cultured to mid-log phase in tryptic soy broth (TSB; Oxoid Ltd, Basingstoke, UK) at 37° C. and 200 rpm, centrifuged for 10 min at 3,000 rpm and then resuspended in brain heart infusion broth (BHI; Oxoid Ltd, Basingstoke, UK). Next, approximately 1×10⁷ CFU bacteria in BHI were cultured in a 96-well flat-bottom polystyrene microplate sealed with breathable seal for 7 days. Thereafter, the biofilms were washed, exposed daily for 3 days to high doses (10× minimal inhibitory concentration, i.e. the lowest concentration that caused a lack of visible growth; MIC) of rifampicin and ciprofloxacin (both from Sigma-Aldrich). Thereafter, the biofilms were washed twice with PBS to remove the antibiotics and bacterial cells in suspension, and then exposed for 24 hrs to increasing concentrations of AM-2 (in PBS supplemented with 1% DMSO) and 1% Tween-80) or to 10×MIC antibiotics as a control. Finally, the released bacteria were removed by washings and the biofilms were sonicated to assess the number of viable bacteria within the biofilms. To take possible slow-growing bacteria into account we analyzed the bacterial plates again after a week. Results are expressed as the number of surviving bacteria (CFU/ml).

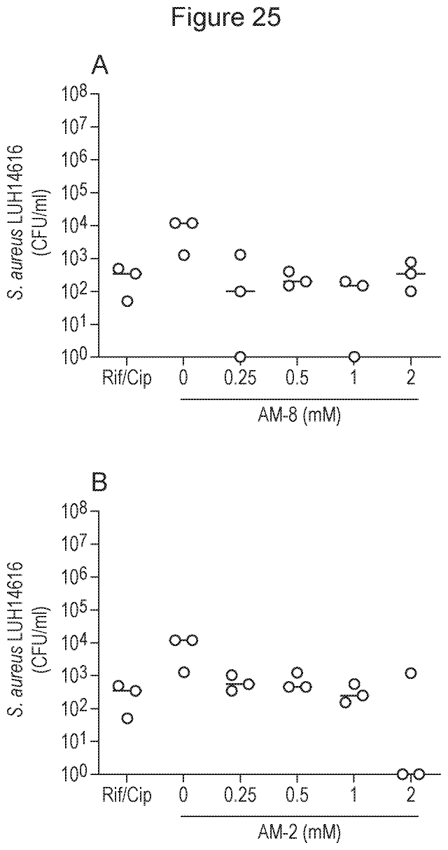

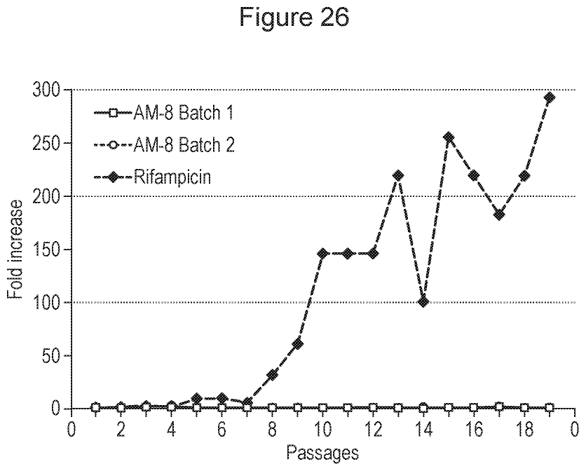

Results. As persisters are hard to eliminate with antibiotics and likely responsible for reinfections (Lewis, 2020), we assessed the effect of AM-2 and AM-8 on persisters within an antibiotics-exposed mature AMR S. aureus biofilm by sonication. Results revealed that AM-2 at the highest dose was effectively against persisters (FIG. 25). It was concluded that AM-2 is effective against bacteria in their persister state. In this state the bacteria are difficult to treat with the classical antibiotics.

### Example 5. Repeated Treatments

Current antibiotics operate primarily through growth-dependent mechanisms and target rapidly-dividing bacteria. However, antibiotic resistance may emerge as result of repeated antibiotic treatment to control persistent and recurring infection. In this example it was demonstrate that no significant antibiotic resistance was built up after repeated exposure of Staphylococcus aureus LUH14616 to diisopropyl thiosulfonate. Scope. In this example the possibility that diisopropyl thiosulfonate (AM-8) induces resistance in AMR Staphylococcus. aureus LUH 14616 was investigated with two independently synthesized batches of material.

Experimental design. Potential resistance to AM-8 was assessed as described by Habets and Brockhurst (2012) (Therapeutic antimicrobial peptides may compromise natural immunity. Biol Lett 8:416-418). Briefly, log phase bacteria (S. aureus LUH 14616) were diluted in RPMI-mod to a concentration of 2×10⁶ CFU/ml. Next, 50 μl of this bacterial suspension were mixed with 50 μl of a range of dilutions of AM-8 or—as comparator-rifampicin in v-bottom 96-wells plates. Thereafter, the plates were covered with a plastic seal and incubated for 24 hrs at 37° C. in a shaking incubator (200 rpm). The plates were then centrifuged for 5 min at 2,000 rpm and the growth inhibition was determined. To start the resistance development testing, 100 μl of a mid-log bacterial suspension containing 2×10⁶ CFU/ml were mixed with 150 μl of RPMI-mod and 5 μl of this diluted bacterial suspension were applied to v-bottom wells containing a dilution range of AM-8 or antibiotics (various concentrations below and above MIC) in RPMI-mod (RPMI 1640 modified with 20 mM Hepes and L-glutamine and without sodium bicarbonate (Sigma-Aldrich); further referred to as RPMI-mod). After 24 hrs at 37° C. in a shaking incubator (200 rpm) the growth inhibition was determined. 100 μl of the bacterial suspension from the 0.5×MIC wells were mixed with 150 μl of RPMI-mod and 5 μl of the diluted bacterial suspension were used to inoculate a new 96-wells plate containing a dilution series of AM-8 or rifampicin, and these mixtures were then incubated as described above. If resistance developed, the concentration range of AM-8 and/or antibiotic was adjusted accordingly. This was repeated 19 times. Results are expressed as the fold-increase in MIC compared to the MIC at the start of the experiment.

Results. The ability of AM-8 to induce resistance in Staphylococcus aureus LUH14616 was assessed for two batches (each in quadruplicate). Results revealed that the MIC for AM-8 hardly increased (a 2-fold increase was seen occasionally) within 19 passages (FIG. 26). For rifampicin there was a rapid increase noticed in the MIC in S. aureus LUH 14616 by 10-fold seen at passages 4-5, after which the MIC further increased to 146-fold after passage 10 and reached >292-fold increase after the last passage. Conclusion. In contrast to rifampicin, AM-8 did not increase the MIC during 19 passages, indicating no resistance development to AM-8 occurs.

### Example 6: Cytotoxicity Tests on Caco-2 and HepG2 Cell Lines

To prevent health damage to infected individuals, cytotoxicity of the antimicrobial compounds that are used to control infections should be acceptable during their application. In this example it is shown that exposure of Caco-2 and HepG2 cell lines to bis(4-chlorophenyl) disulfide, bis(4-fluorophenyl) disulfide, bis(4-chlorophenyl) thiosulfonate and diisopropyl thiosulfonate have cytotoxic effects on these cell lines.

Scope. As standard tests cytotoxicity is measured on Caco-2 and HepG2 cell lines. Cytotoxicity of mentioned cell lines was determined by measuring the amount of converted MTT (3-(4,5-dimethylthiazol-2-yl)-2,5-diphenyltetrazolium bromide) by a mitochondrial dehydrogenase after exposure of various concentrations of the antimicrobial compounds. In living cells the dehydrogenase converts the soluble MTT into water insoluble purple formazan crystals that can not pass the cell membrane. The higher the metabolic activity of the cell, the more purple crystals are formed, the lower the cytotoxicity of the antimicrobial compound.

Experimental design. The Caco-2 cell line as colorectal adenocarcinoma cells and HepG2 (derived from human liver) were used for the current experiments and were obtained from the ATCC. The intestinal Caco-2 cell line was cultured in 75-cm² culture flasks. Minimum Essential Medium (MEM) (Catalog Number(s) 31095029, ThermoFischer Scientific), supplemented with 10% (v/v) inactivated fetal calf serum (FCS) (Gibco), 1% (v/v) nonessential amino acids (Gibco), 1% sodium pyruvate (Gibco) and penicillin (100 U/ml)/streptomycin (100 μg/ml.) was utilized as medium for the growing cells. The cells were preserved in an incubator to provide them with the optimum moisture and temperature (humidified atmosphere of 95% air and 5% CO₂ at 37° C. for 72 hours). The Caco-2 cells were seeded in flat bottom 96-well plates for performing the experiments.

HepG2 adherent cells were isolated from human liver and show epithelial morphology. Culture condition of the cells were followed based on the standard protocols. HepG2 cell line was cultured in 75-cm² culture flasks. Dulbecco's modified Eagle's minimum essential medium (DMEM)+Glutamax, supplemented with 10% (v/v) inactivated fetal calf serum (FCS) (Gibco), and penicillin (100 U/ml.)/streptomycin (100 μg/ml.) was utilized as medium for the growing cells. The cells were preserved in an incubator to provide them with the optimum moisture and temperature (humidified atmosphere of 95% air and 5% CO₂ at 37° C. for 72 hours).

The cells were grown on flat bottom 96-well plates to the appropriate density to run the experiments. The cells were exposed to increasing concentrations of emulsified test compound (8 concentration, ranging from 7.8 μM to 1 mM) in standard culture medium (Catalogue 31331028, ThemoFischer Scientific) for each cell line. It should be noted that all the experiments were conducted in FBS (Catalog A3840101, ThermoFischer Scientific) and antibiotic free medium. The concentration of Tween-80 and DMSO that is present in the assay to emulsify the antimicrobial compound was in the range from 0,007% to 1%. The cells were incubated for 24 h with emulsified test compound and the assay was performed based on the standard protocol. By the end of the experiment the medium (cell supernatants) was removed and the lysis buffer (0.04 M hydrochloric acid, in propan-2-ol) was added to each well to solubilize the purple formazan crystals. As the final step, the absorbance was recorded by a multi-well reader at 595 nm. Control experiment with emulsifiers Tween 80 and DMSO but without antimicrobial compounds was performed in a same way on both cell lines. The results from all experiments were expressed as mean #standard error of the mean (SEM) conducted in triplicate (three wells/condition) of three independent experiments. Differences between results were statistically evaluated using One-way ANOVA with Bonferroni post-hoc test.

### Results.

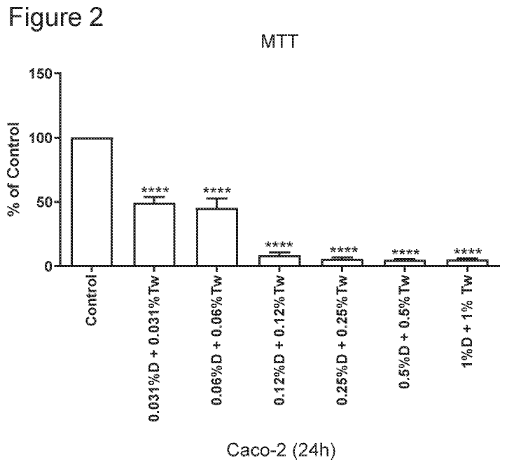

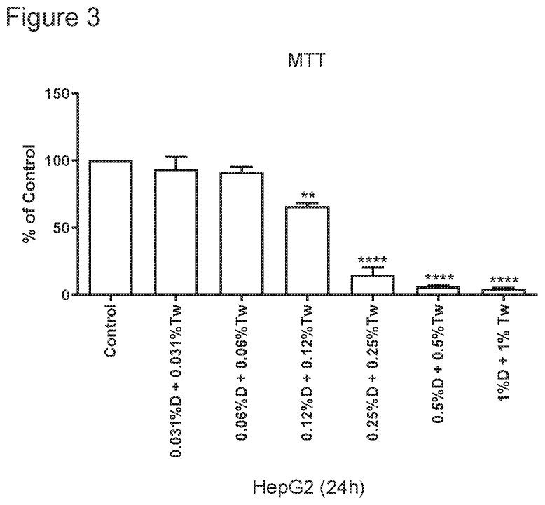

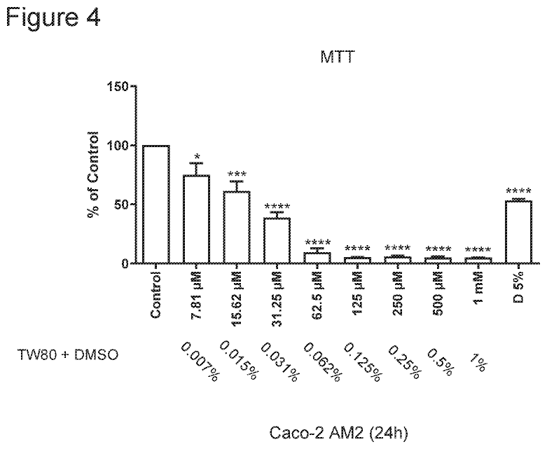

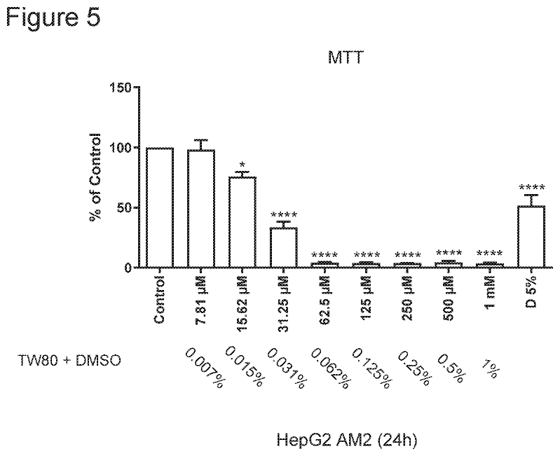

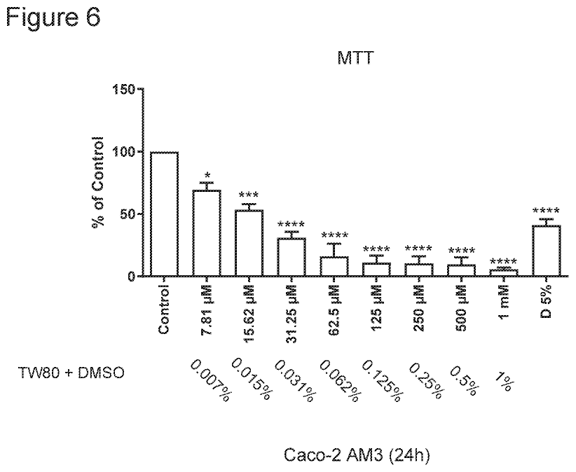

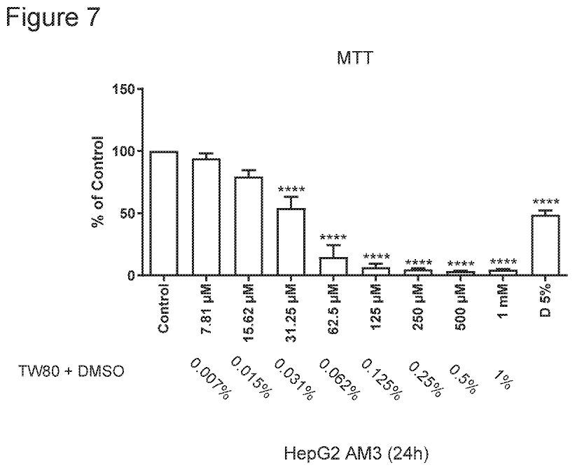

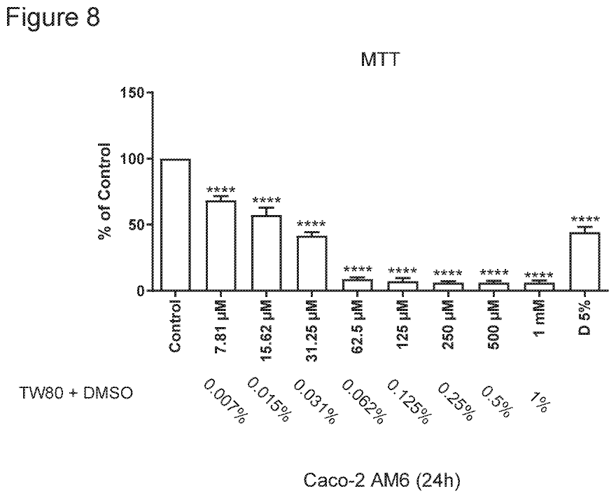

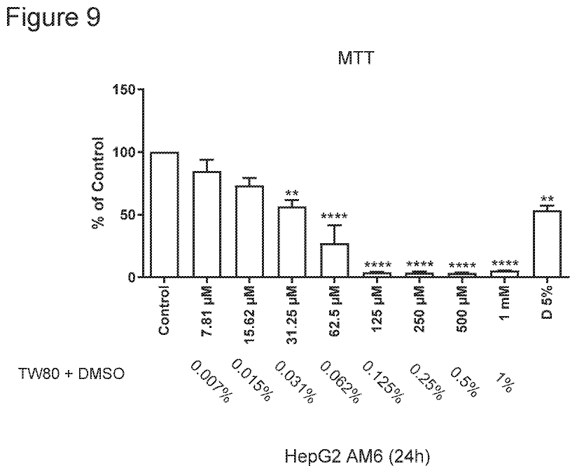

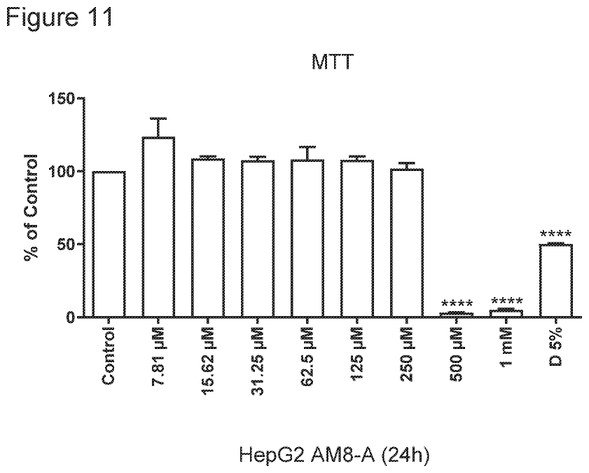

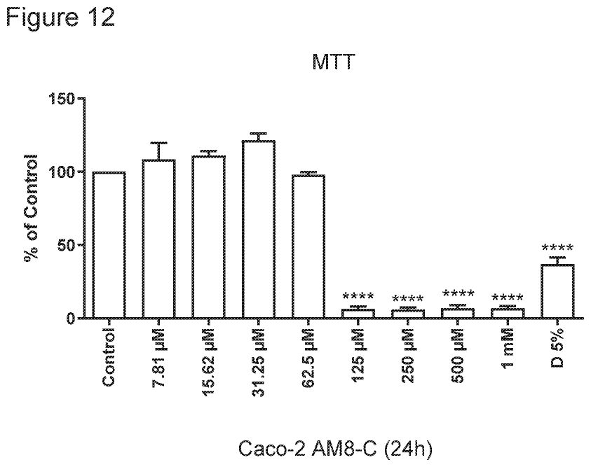

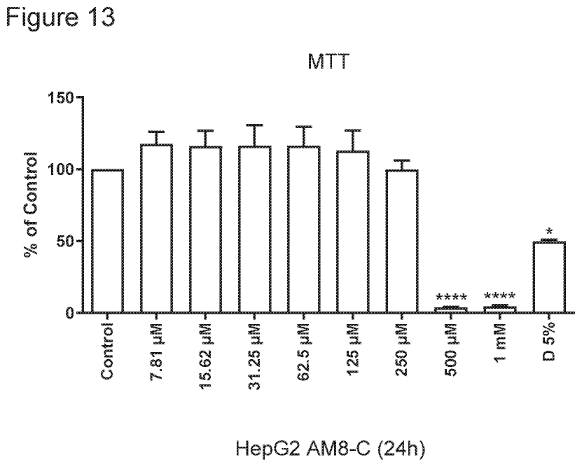

In FIGS. 2-3 a control experiment was performed on both cell lines (Caco-2 and HepG2), in which the cytotoxic effects of the applied concentrations of emulsifiers Tween 80 and DMSO were tested, without the Antimicrobial (AM) compounds. Exposure with the increasing concentrations of emulsifiers on the Caco-2 and HepG2 cell lines showed cytotoxic effect. In FIGS. 4-13 the effects of the emulsified antimicrobial compounds on cytotoxicity of the Caco-2 and HepG2 cell lines are presented. DMSO 5% (marked as D 5%) was used as the positive control for cytotoxicity. The lowest X-axis represent the concentration Tween-80 and DMSO that is present in the assay to emulsify the antimicrobial compound, ranging from 0,007% to 1%. The following antimicrobial compounds were tested and are marked as: AM2, bis(4-chlorophenyl) disulfide; AM3, bis(4-fluorophenyl) disulfide; AM6, bis(4-chlorophenyl) thiosulfonate, and AM8, diisopropyl thiosulfonate. AM8-A and AM8-C correspond to two separate batches of synthesized AM8.

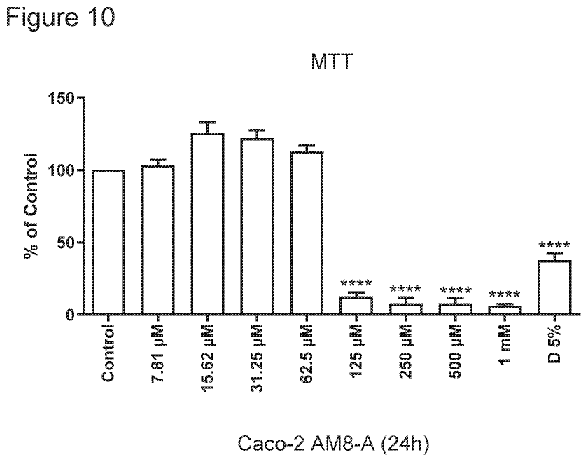

Since the emulsifiers also showed a cytotoxic effect that counts up to the cytotoxic effect of the antimicrobial, the effects of the emulsifiers (FIGS. 2-3) should be withdrawn from the total cytotoxic effect. Exception is AM8 (diisopropyl thiosulfonate, FIGS. 10-13): as this antimicrobial compound hardly needs any emulsifier. In particular, no DMSO and 30 times less tween-80, as compared to the other AM compounds, was used to emulsify AM8.

Based on the obtained results, it is concluded that all compounds showed a slight cytotoxic effect and that cytotoxicity is lowest when the Caco-2 and HepG2 cells are exposed to diisopropyl thiosulfonate (AM8, FIGS. 10-13).

### Example 7: Treatment of Infected Plant Crops

### Example 7a

In this example the antimicrobial activity of the compounds of the invention on plants and plant parts is tested.

For this purpose, plants from the group consisting of Begoniaceae, Solanaceae, Amaranthaceae, Rosaceae and Brassicaceae are selected. Infection caused by various bacteria and fungi are tested. In particular, the antimicrobial activity of the compounds of the invention is tested against pathogenic fungi selected from Pythium ultimum, Fusarium oxysporum, Fusarium solani, Phytophthora cactorum, Rhizoctonia solani; and against pathogenic bacteria selected from Xanthomonas campestris, Erwinia amylovora, Rhizobium spp., Clavibacter michiganensis, Agrobacterium radiobacter. An exemplary overview of the infected plants is given in Table 11 below.

TABLE 11
Exemplary overview of plants infected
with fungi/bacteria to be tested

| Fungi/Bacteria | Plant |
|---|---|
| Pythium ultimum | Begonia |
| Fusarium oxysporum | Tomato, Potato |
| Fusarium solani | Sugar beet |
| Phytophthora cactorum | Strawberries |
| Rhizoctonia solani | Potato |
| Xanthomonas campestris | Cabbage |
| Erwinia amylovora | Apple |

### Example 7b. Inhibition of Fungal and Bacterial Plant Pathogens by Antimicrobial Compounds

Many crops of the group Begoniaceae, Solanaceae, Amaranthaceae, Rosaceae and Brassicaceae are infected by plant pathogens of the fungal genus Pythium, Fusarium, Phytophthora, Rhyzoctonia and bacterial genus Xanthomonas, Erwinia, Rhizobium, Clavibacter and Agrobacterium. In this example, it was shown that growth of fungal and bacterial plant pathogens is inhibited by the mentioned antimicrobial compounds and act as effective control agents.

Scope. In this example the antimicrobial activity of the compounds of the invention is tested on plant pathogens. Di-n-propyl thiosulfonate (QQ2) was used as a reference compound.

In particular, the antimicrobial activity of bis(p-chlorophenyl) disulfide (AM2), bis(p-fluorophenyl) disulfide (AM3), bis(p-chlorophenyl) thiosulfonate (AM6) and diisopropyl thiosulfonate (AM8) of the invention is tested against pathogenic fungi selected from Pythium aphanidermatum, Phytophthora cinnamiomi, Fusarium oxysporum, Sclerotinia sclerotiorum, Rhizoctonia solani and Botrytis cinerea.

Experimental design. The effects of mentioned compounds on growth of the fungi were tested on agar plates with various dilutions of the compounds. The fungus was inoculated in the middle of the agar plate. At various time intervals the diameter of the colony was measured.

The fungi are pregrown on the indicated agar media as indicated in the table 12.

TABLE 12
List of fungi that were tested in this example. PDA, potato dextrose
agar medium; OA, oatmeal agar; MEA, malt extract agar medium

| | Test | |
|---|---|---|
| Pathogen | Medium | Remarks |
| Pythium aphanidermatum | PDA | Certify collection: isolated |
| | | from Chrysanthemum, identified |
| | | by DNA sequencing |
| Phytophthora cinnamiomi | PDA | CBS 277.55 |
| Fusarium oxysporum | PDA | Certify collection: isolated |
| | | from spinach, identified |
| | | by DNA sequencing |
| Sclerotinia sclerotiorum | OA | Certify collection: isolated |
| | | from Cichory |
| Rhizoctonia solani | MEA | CBS 323.84 |
| Botrytis cinerea | OA | Certify collection: isolated |
| | | from tomato |

The fungi were pregrown on appropriate agar media, for example potato dextrose agar or malt extract agar at 24 C. After 3, 7, 10 and 14 days of incubation at 24 C, the diameter of the fungal colonies were measured on the agar plates. Since the agar plates have a diameter of 8 cm, this was the maximum colony size.

Agar media were prepared with various dilutions of bis(p-chlorophenyl) disulfide (AM2), bis(p-fluorophenyl) disulfide (AM3), bis(p-chlorophenyl) thiosulfonate (AM6) and diisopropyl thiosulfonate (AM8).

The test compounds were added to the agar media when the liquid agar medium was still hand-warm to prevent heat-dependent decomposition of the compounds. 50-fold concentrated stock solutions of the antimicrobial compounds of each dilution were prepared in 50% tween-80 and 50% DMSO (in this way the concentrations DMSO and tween 80 were the same in all agar plates). The following concentrations of the antimicrobial compounds were used for each strain in agar plates: 1.0 mM, 0.50 mM, 0.25 mM, 0.125 mM and 0.063 mM in the corresponding medium as indicated in table 12. Each concentration was tested in 4-fold in 8 cm diameter petridishes and each dish contained 11 ml agar medium.

Hereafter, the plates were inoculated in the middle of the dish. Incubation took place at 24 C. Colony diameters were measured at t=3, 7, 10 and 14 days after inoculation. Since the diameter of the petri dishes are 8 cm this diameter was the maximal diameter that was measured.

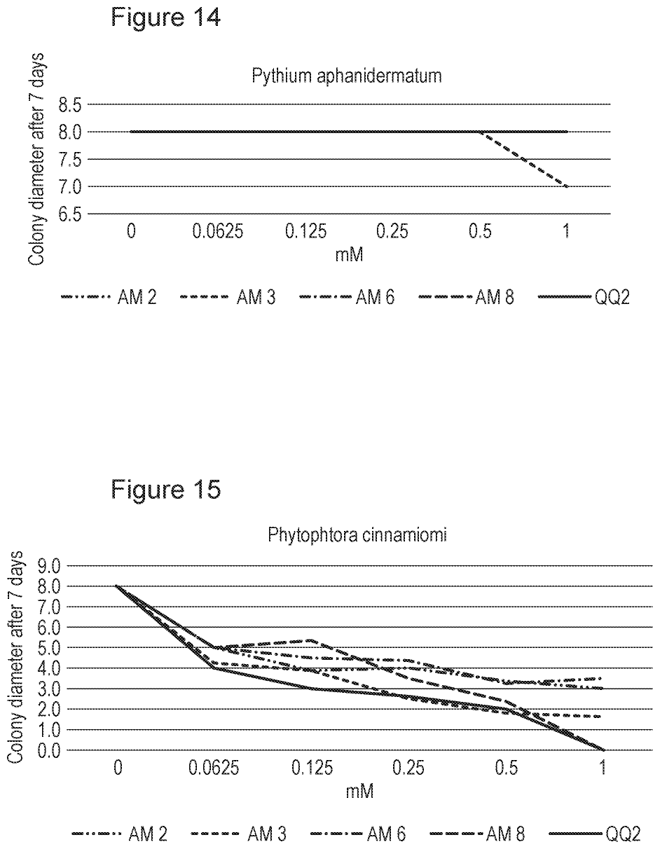

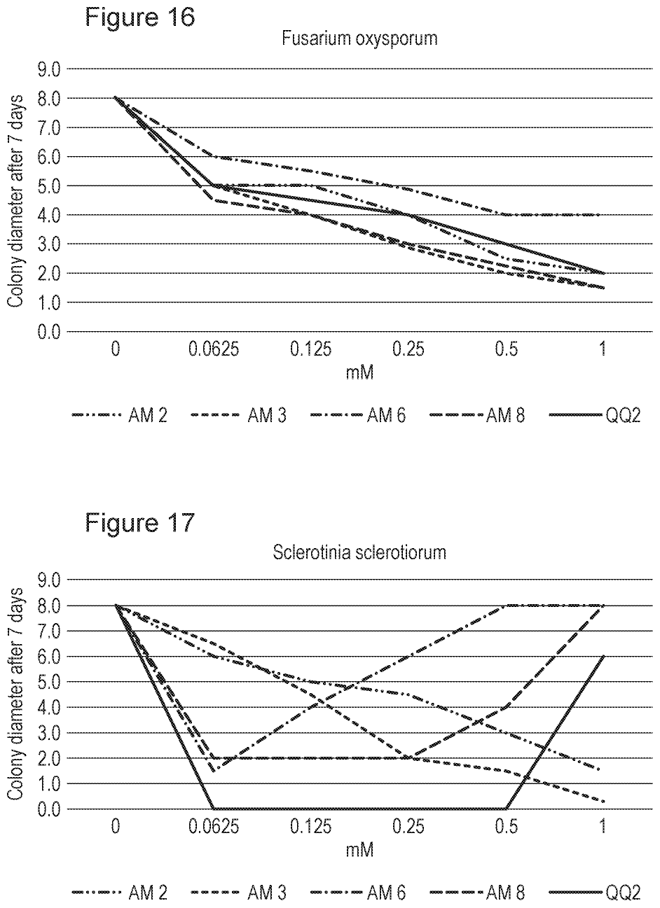

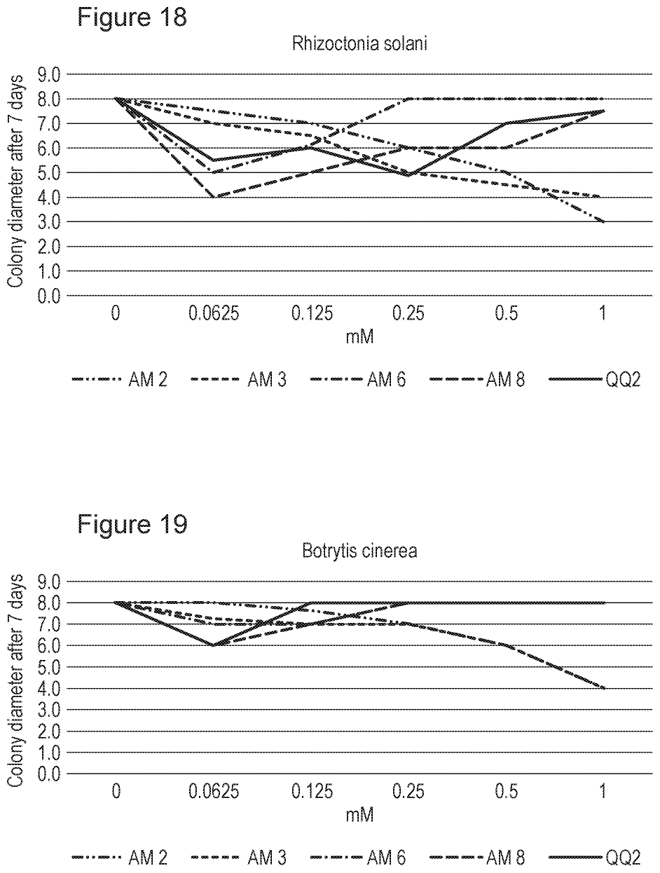

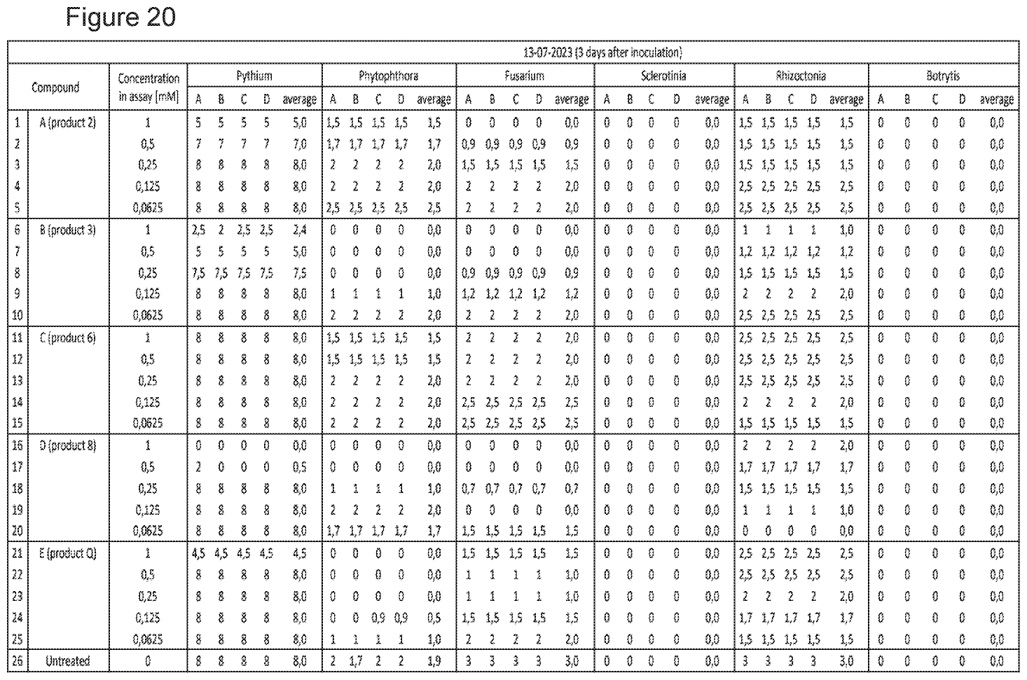

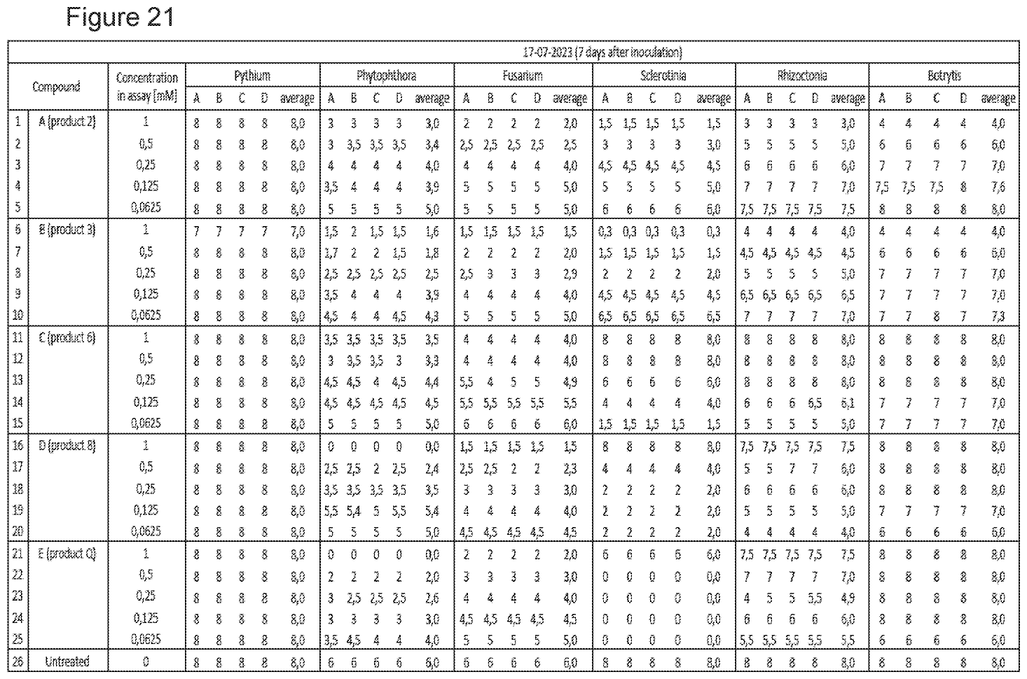

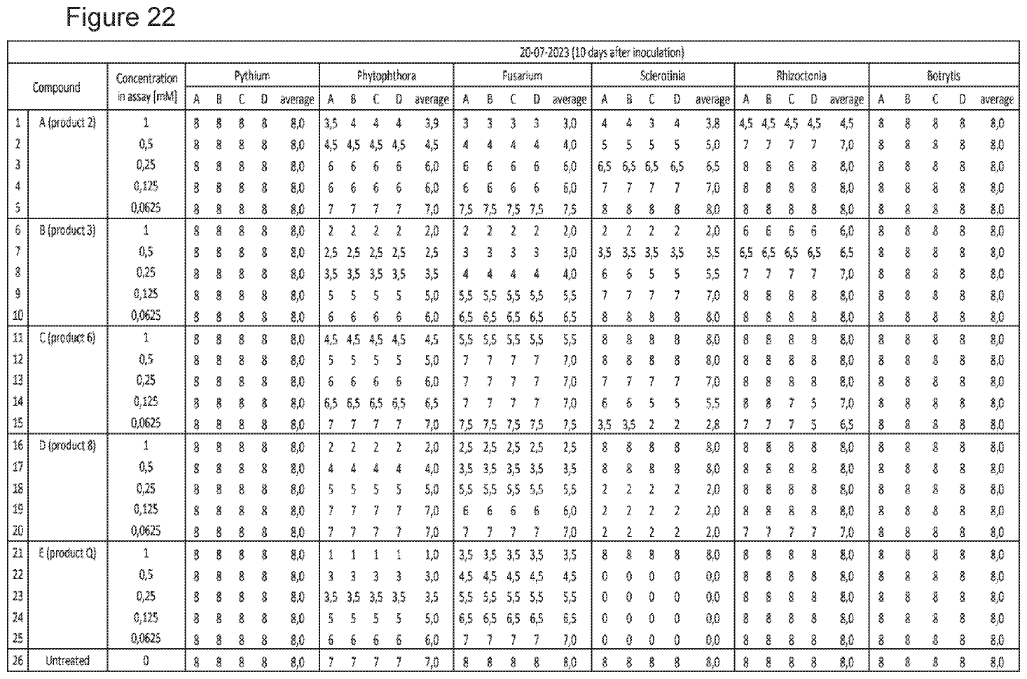

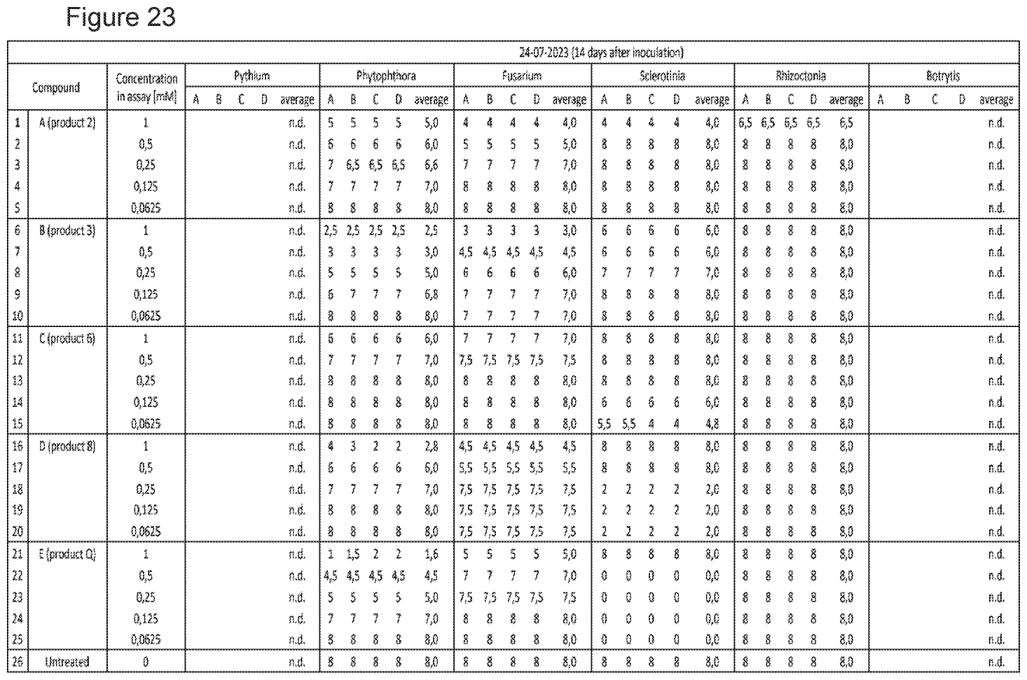

Results. In FIGS. 14-19 the effects of the various antimicrobial compounds are presented after 7 days of incubation: at day 7 of incubation, differences with respect to growth inhibition are clearly demonstrated. The larger the colony diameter, the less growth inhibition was shown. Pythium (FIG. 14) did not show any significant growth at the tested concentrations of the antimicrobial compounds at day 7 after inoculation (although growth inhibition is observed at earlier time points), whereas growth of the other fungi was inhibited. Phytophthora cinnamiomi (FIG. 15), Fusarium oxysporum (FIG. 16) and Sclerotina sclerotiorum (FIG. 17) were most sensitive for the antimicrobial compounds. Furthermore, growth inhibition was dependent on the applied antimicrobial compound and the fungus that it was applied to. Additionally, growth inhibition of Phytophthora cinnamiomi and Fusarium oxysporum was concentration dependent. In particular, higher concentration of the antimicrobial compound showed bigger growth inhibition (FIGS. 15-16). The raw data of the experiment at t=3, 7, 10 and 14 is presented in FIGS. 20-23. The tested antimicrobials are marked as follows: product 2, bis(4-chlorophenyl) disulfide (AM2); product 3, bis(4-fluorophenyl) disulfide (AM3); product 6, bis(4-chlorophenyl) thiosulfonate (AM6); product 8, diisopropyl thiosulfonate (AM8); and product Q, di-n-propyl thiosulfonate (QQ2).

Conclusion. In this example it is clearly shown that growth of plant pathogens is clearly inhibited by bis(p-chlorophenyl) disulfide, bis(p-fluorophenyl) disulfide, bis(p-chlorophenyl) thiosulfonate and diisopropyl thiosulfonate and this shows the applicability in microbial pest control in crops. In table 13 some examples are presented with respect to pest in plants and crops that could be treated in case the pests occur.

TABLE 13
Exemplary overview of plants that are subject to the tested fungi.

| Fungi/Bacteria | Plant |
|---|---|
| Pythium ultimum | Begonia |
| Fusarium oxysporum | Tomato, Potato |
| Fusarium solani | Sugar beet |
| Phytophthora cactorum | Strawberries |
| Rhizoctonia solani | Potato |
| Xanthomonas campestris | Cabbage |
| Erwinia amylovora | Apple |

1. (canceled)

2. A method of treating or preventing a microbial infection in an individual in need thereof comprising administering to the individual a compound selected from Bis(4-fluorophenyl) disulfide, Bis(4-chlorophenyl) disulfide, Bis(4-chlorophenyl) thiosulfonate, Bis(4-fluorophenyl) thiosulfonate, Bis(4-chlorophenyl) thiosulfinate, Bis(4-fluorophenyl) thiosulfinate, and Di-isopropyl thiosulfonate, or a composition comprising at least one said compound.

3. A composition comprising a compound selected from Bis(4-fluorophenyl) disulfide, Bis(4-chlorophenyl) disulfide, Bis(4-chlorophenyl) thiosulfonate, Bis(4-fluorophenyl) thiosulfonate, Bis(4-chlorophenyl) thiosulfinate, Bis(4-fluorophenyl) thiosulfinate, and Di-isopropyl thiosulfonate, wherein the composition is a pharmaceutical composition, a pesticide, a foodstuff composition a cleaning composition, a disinfecting composition, or an agricultural composition.

4. An article having a surface at least partially coated with the composition.

5. The composition according to claim 3, wherein the composition is a cleaning or disinfecting composition and further comprises a surfactant.

6. An in vitro method comprising applying to a surface the composition according to claim 5.

7. The method according to claim 6, wherein said method is a method of sanitizing or disinfecting said surface.

8. (canceled)

9. A cleaning or disinfecting product comprising the composition according to claim 3.

10. The composition according to claim 3, wherein the composition is an agricultural composition and further comprises an agricultural acceptable excipient, carrier and/or solvent.

11. A method of preventing or treating an infection on a plant or plant part, comprising contacting the plant or plant part with a compound selected from Bis(4-fluorophenyl) disulfide, Bis(4-chlorophenyl) disulfide, Bis(4-chlorophenyl) thiosulfonate, Bis(4-fluorophenyl) thiosulfonate, Bis(4-chlorophenyl) thiosulfinate, Bis(4-fluorophenyl) thiosulfinate, and Di-isopropyl thiosulfonate or a composition comprising at least one said compound such that an infection is prevented or treated.

12. (canceled)

13. The method according to claim 11, wherein said compound or composition is applied directly to said plant, to the seed of said plant or to the soil of said plant or seed.

14. The method according to claim 11, wherein the plant is selected from the group consisting of Begoniaceae, Solanaceae, Amaranthaceae, Rosaceae and Brassicaceae, preferably wherein the plant is selected from the group consisting of Begonia, Tomato, Potato, Sugar beet, Strawberries, Cabbage, Apples, Orchidaceae, Chrysanthemum, Fabaceae, Cucurbitaceae, Pisum, Vitis, Vaccinia and Lactuca.

15. The article according to claim 4, wherein the article is a medical device or surgical device.
